(19) United States
(12) Patent Application Publication
(10) Pub. No.: US 2026/0201015 A1
(43) Pub. Date: Jul. 16, 2026

(54) DE NOVO DESIGNED PROTEIN BINDERS TO NATIVE FLEXIBLE HELICAL PEPTIDES

(71) Applicant: UNIVERSITY OF WASHINGTON, Seattle, WA (US)

(72) Inventors: David BAKER, Seattle, WA (US); Susana VAZQUEZ-TORRES, Seattle, WA (US); Isaac LUTZ, Seattle, WA (US); Philip LEUNG, Seattle, WA (US); Preetham VENKATESH, Seattle, WA (US); Joseph L. WATSON, Seattle, WA (US); David JUERGENS, Seattle, WA (US); Nathaniel BENNETT, Seattle, WA (US); Hsien-Wei YEH, Seattle, WA (US); Michael J. MACCOSS, Seattle, WA (US); Andrew Norbert HOOFNAGLE, Seattle, WA (US); Huu H. HUYNH, Seattle, WA (US); Eric HUANG, Seattle, WA (US); Gyu Rie LEE, Seattle, WA (US); Jessica BECKER, Seattle, WA (US)

(21) Appl. No.: 19/129,807

(22) PCT Filed: Dec. 1, 2023
(86) PCT No.: PCT/US2023/082112

§ 371 Date: May 14, 2025

Related U.S. Application Data
(60) Provisional application No. 63/430,545, filed on Dec. 6, 2022.
(60) Provisional application No. 63/520,162, filed on Aug. 17, 2023.

(51) Int. Cl. C07K 14/72; A61K 38/00; G01N 33/74

(52) U.S. Cl. CPC C07K 14/72; G01N 33/74; A61K 38/00

(57) ABSTRACT

Target-binding polypeptide are disclosed that include an amino acid sequence at least 50% identical to the amino acid sequence selected from the group consisting of SEQ ID NO:1-12, nucleic acids encoding such polypeptides, and methods for use of the polypeptides in detecting binding of the target and correlating binding to the target with a disease state.

### CROSS REFERENCE

This application claims priority to U.S. Provisional Patent Application Ser. No. 63/430,545 filed Dec. 6, 2022 and 63/520,162 filed Aug. 17, 2023, each incorporated by reference herein in its entirety.

### FEDERAL FUNDING STATEMENT

This invention was made with government support under Grant No. 1 K99 EB 031913-01A1, awarded by the National Institute of Biomedical Imaging and Bioengineering and Grant No. 5U19AG065156-03, awarded by the National Institute on Aging. The government has certain rights in the invention.

### SEQUENCE LISTING STATEMENT

A computer readable form of the Sequence Listing is filed with this application by 20 electronic submission and is incorporated into this application by reference in its entirety. The Sequence Listing is contained in the file created on Nov. 28, 2023 having the file name “23-0947-WO.xml” and is 16,740 bytes in size.

### BACKGROUND

Many peptide hormones form an alpha-helix upon binding their receptors, and sensitive detection methods for them could contribute to better clinical management of disease. The design of interactions between proteins and short peptides with helical propensity is an unmet challenge.

### SUMMARY

The present disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-12. In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1, 2, and 10, wherein the polypeptide binds to parathyroid hormone (PTH). In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:3 and 8, wherein the polypeptide binds to glucagon (GCG).

In a further embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:4 and 9, wherein the polypeptide binds to neuropeptide Y (NPY). In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:5, wherein the polypeptide binds to secretin (SCT). In a further embodiment the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:6, wherein the polypeptide binds to gastric inhibitory peptide (GIP).

In one embodiment the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7, wherein the polypeptide binds to glucagon-like peptide 1 (GLP1). In another embodiment the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:11, wherein the polypeptide binds to BIM BH3 peptide (Bim). In a further embodiment the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:12, wherein the polypeptide binds to peptide YY (PYY).

The disclosure also provides nucleic acids encoding the polypeptide or fusion protein of any embodiment herein, expression vectors comprising the nucleic acid operatively linked to a suitable control sequence, host cells comprising the polypeptide, fusion protein, nucleic acid, or expression vector of any embodiment, and pharmaceutical composition, comprising (a) the polypeptide, fusion protein, nucleic acid, expression vector, and/or host cell of any embodiment; and (b) a pharmaceutically acceptable carrier. The disclosure also provides methods, comprising contacting a biological sample with the polypeptide or fusion protein of any embodiment, and detecting binding of the polypeptide or fusion protein with target present in the biological sample, wherein presence of the biological sample correlates with a disease state.

### DESCRIPTION OF THE FIGURES

FIG. 1. Design strategies for binding helical peptides. (a) Parametric approach. Left: sampling groove scaffolds varying supercoiling and helix distance to fit different targets. Middle: design model (spectrum) and PTH target of the best parametrically designed PTH binder. Right: Split NanoBiT titration of PT- and the binder showed weak binding. (b) Inpainting binder optimization. Left: Redesign of parametrically generated binder designs using RFjoint inpainting to expand the binding interface and ProteinMPNN to redesign the sequences. Middle: AF2 prediction of inpainted design (spectrum) with extended interface, and PTH target. Right: FP measurements indicate 6.1 nM binding to PTH and weak binding to off-target PTHrp. Weak Binder Sequence (Left) SEQ ID NO: 14 & Interface-Expanded Sequence (Right) SEQ ID NO: 15. (c) Threading approach to peptide binder design. Left: Starting with a helix-bound scaffold, a target is threaded into the bound helix and the interface is redesigned. Middle: AF2 prediction of design (spectrum) and SCT target. Right: FP measurements indicate 3.95 nM binding to SCT and 12 nM binding to GCG.

FIG. 2. Peptide binder optimization with RF Diffusion: (a) Partially diffused binders to NPY and GCG. Top: Design model (spectrum) of partially diffused binder to NPY and FP measurements indicate a 5.29 nM binding affinity to NPY target and selectivity over PYY. Bottom: Design model (spectrum) of partially diffused binder to GCG and FP measurements indicate a subnanomolar binding affinity to GCG and selectivity over SCT. (b) Left: AF2 model aligned at 0.72 Å RMSD with the 1.95 Å crystal structure of the best inpainted GCG binder. Right: AF2 model aligned at 0.6 Å RMSD with the 1.81 Å crystal structure of the best partially diffused GCG binder. (c) Comparison of the crystal structure of the inpainted GCG binder with the crystal structure of the partially diffused GCG binder; while the two structures have considerable topological similarity, there are many small readjustments. FP titrations with GCG indicate much tighter binding for the partially diffused GCG binder. (d) Left: zoom in view of the original inpainted GCG binder. Design overlaid with the crystal structure shows the interaction of Phe 13 with the target surface. Middle: zoom in view of the partially diffused GCG binder. Design overlaid with the crystal structure shows the interaction of Ile 13 with the target surface with better shape complementarity. Right: mutating the original inpainted design model to lie at position 13 does not restore the favorable interaction in the partially diffused design because the backbone is in a different position.

FIG. 3. Peptide binder design with RF Diffusion: (a) Design of picomolar affinity PTH binder. Left: Design model of PTH binder (spectrum, AF2 metrics in Supplementary Table 3). Middle: Circular Dichroism (CD) data shows that the binder has helical secondary structure and is stable at 95° C. (inset). Right: FP measurements with PTH indicate a sub-nanomolar binding affinity and no binding for PTHrp indicates high specificity. (b) Design of picomolar affinity Bim binder. Left: Design model of Bim binder (spectrum, AF2 metrics in Supplementary Table 3). Middle: CD data shows that the binder has helical secondary structure and is stable at 95° C. (inset). Right: FP measurements with Bim indicate a sub-nanomolar binding affinity. (c) Crystal structure of Bim binder. Top inset: a kinked helix in the diffused backbone accommodates Arg 13 of Bim. Middle: crystal structure of Bim. Bottom inset: A cross-interface hydrogen bond network formed between Asn 20 of Bim and Thr 73 and Asn 77 of the binder. (d) RF Diffusion_flex with PYY sequence input. Left: PYY in complex with its native Neuropeptide Y Y2 receptor³⁹ (PDB ID: 7YON) shows flexibility at its N- and C-terminus. Middle: design model of the binder (spectrum) with PYY target. The C-terminus is fully structured while the N-terminus shows increased orderedness. Right: FP measurements with PYY indicate a 24.5 nM binding affinity.

FIG. 4. Application of designed binders to sensing and detection. (a) Protein biosensors for PTH detection. The PTH lucCage biosensor, depicting the cage and latch (left), key (right), and the PTH binder, thermodynamically shifts from OFF state to an ON state in the presence of PTH peptide target. This conformational change brings two luciferase halves close in proximity and the active luciferase is reconstituted to generate luminescence. (b) Left: titration of PTH results in luminescence increase. Middle: response of lucCageFH biosensor at the linear concentration range, indicating a 10 nM limit of detection (see methods). Right: titration curve of 10 nM lucCagePTH+lucKey to various concentrations of PTH. (c-d) The designed PTH binder enables robust recovery of PTH from complex mixtures. (c) Enrichment experiment schematic. (d) LC-MS/MS chromatograms for SVSEIQLMHNLGK (SEQ ID NO: 16), the N-terminal tryptic peptide of PTH: different peptide fragments detected by the LC-MS/MS assay are in different colors (left). Mean chromatographic peak areas for triplicate measurements of each sample type. Error bars represent standard deviation (right).

FIG. 5. Comparison of binding affinities between the PTH inpainted binder (SEQ ID NO 2) and the parametrically redesigned binder using ProteinMPNN only. Structural extension with inpainting and ProteinMNN sequence redesign resulted in a significant increase in the binding affinity of the parametrically designed PTH binder. Conversely. ProteinMNN redesign performed solely on the original parametrically designed binder did not lead to an increase in affinity.

FIG. 6. Weak inpainted binders for NPY and GCG using extended parametric designs. (a) Left: Design model of the tightest GCG binder (SEQ ID NO 8). Right: FP titration for the tightest GCG binder indicates ~231 nM binding affinity. (b) Left: Design model of the tightest NPY binder (SEQ ID NO 9). Right: FP titration for the tightest NPY binder indicates 3.5 μM binding affinity.

FIG. 7. Additional binders made using threading and redesign. (a) Left: Design model of the tightest GLP1 binder (SEQ ID NO 7). Right: FP titration for the tightest GLP1 binder indicates 68.8 nM binding affinity. (b) Left: Design model of the tightest GIP binder (SEQ ID NO 6). Right: FP titration for the tightest GIP binder indicates 6.96 nM binding affinity.

FIG. 8. SEC traces of peptide binders. (a) SEC reinjection of the most abundant monodisperse peak for the inpainted PTH binder (SEQ ID NO 2). (b) SEC reinjection of the most abundant monodisperse peak for the inpainted GCG binder (SEQ ID NO 3). (c) SEC reinjection of the most abundant monodisperse peak for the inpainted NPY binder (SEQ ID NO 4). (d) SEC reinjection of the most abundant monodisperse peak for the threaded SCT binder (SEQ ID NO 5). (e) SEC reinjection of the most abundant monodisperse peak for the partially diffused GCG binder (SEQ ID NO 8). (f) SEC reinjection of the most abundant monodisperse peak for the partially diffused NPY binder (SEQ ID NO 9). (g) SEC reinjection of the most abundant monodisperse peak for the fully diffused PTH binder (SEQ ID NO 10). (h) Initial SEC of the fully diffused Bim binder (SEQ ID NO 11). (i) SEC reinjection of the most abundant monodisperse peak for the flexibly diffused PYY binder (SEQ ID NO 12).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M.P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} (R.I. Freshney, 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, “about” means+/−5% of the recited value.

All embodiments of any aspect of the disclosure can be used in combination, unless the context clearly dictates otherwise.

Unless the context clearly requires otherwise, throughout the description and the claims, the words ‘comprise’. ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used herein, the amino acid residues are abbreviated as follows: alanine (Ala; A), asparagine (Asn; N), aspartic acid (Asp: D), arginine (Arg; R), cysteine (Cys: C), glutamic acid (Glu; E), glutamine (Gln; Q), glycine (Gly: G), histidine (His; H), isoleucine (Ile; I), leucine (Leu; L), lysine (Lys; K), methionine (Met; M), phenylalanine (Phe: F), proline (Pro: P), serine (Ser; S), threonine (Thr; T), tryptophan (Trp; W), tyrosine (Tyr: Y), and valine (Val: V).

Any N-terminal amino acids are optional, and may be deleted.

In various embodiments, 1, 2, 3, 4, or 5 amino acids may be deleted from the N-terminus and/or the C-terminus of any of the polypeptides disclosed herein.

In one aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-12. As disclosed in the examples that follow, the polypeptides bind specific peptide targets (identified in Table1) with nanomolar to picomolar affinity using the assay described in the examples, and can be used, for example, in various diagnostic assays in which the target can be detected to assess a disease state. Table 2 below shows key residues for binding to the target, and uses for the different peptide binders. The target-interacting residues noted in Table 2 were identified by computationally testing the effect of mutating that residue to every other residue. These residues are primarily at the interface, while some may be long distance interactions.

TABLE 1
Amino acid sequences of peptide binders

| SEQ ID | | Computational | |
|---|---|---|---|
| NO | Target | method | Binder amino acid sequence |
| 1 | PTH | Parametric | SQELIEELIKLAKELAEIKDEEERRKIKRELER |
| | | design | LAEELKEAPASSLLRALALLVIALALIQAAESE |
| | | | EERERARELLERLEELLRELEKQITDERFKEIL |
| | | | RELEELAKELKKQL |
| 2 | PTH | Inpainting | SFELLEKLIELSKELYEVAKKYIETGDPELKKK |
| | | | LEEILKKIEEAYKELLESDAHPLEKALAKLILA |
| | | | EAYVVKSFAYISSGKDLEKAQEYLDKAKEILEE |
| | | | LLKLLEELKKEETDPEKLEIIEELEKIAEELLK |
| | | | EIEE |
| 3 | GCG | Inpainting | MLDELFSLLNKMFELSDKYRELRKELRKAIESG |
| | | | APEEELRELLEKMLEIAKKLLELTKELKKLVED |
| | | | VLKNNPDPVERAKAVLLYAVGVHILYSESSELE |
| | | | VIAERLGFKDIAEKAKEIADKARELKEEVKRKL |
| | | | REIREEVPDPEIRKAAEEAIEMLESNDKRLKEF |
| | | | RKL |
| 4 | NPY | Inpainting | GAAEKLAELYEKFKALREKALEVLKKAVEALEN |
| | | | KADKETLLKLIKELKELAEKFEELAEEFERNAG |
| | | | ESTTASLNATAAYMARIGLLAALLALAKAAGVP |
| | | | EEELEEIKRRIEETAKRAIEAAERLKALAEARG |
| | | | DTKHVAVGVEAVRMATELYELAQKIIDAF |
| 5 | SCT | Threading | SEELEERLREARERLEEARERLEEAREEGDLRE |
| | | | MAPALLEEAPAVLEIARVAAEAGDDEALREAAR |
| | | | RAGEVIRRAGEVGLRAAEEGDTETIREAMLAIL |
| | | | EAQRASAVIALHLARDDPEVAEALRVIERLLRT |
| | | | AERALREGQLEVARLATEAVEALADAILRAREI |
| | | | GRPELVREAARLAEEARRLLEAALEALRAGDEE |
| | | | GARERLARARELIREIRERVRRA |
| 6 | GIP | Threading | SPKEKAERLIKEAKEAAEKAKEAAERSGLEEAK |
| | | | KAAEELTKLLEEAAARVAADPEDETKLRALEKI |
| | | | VEAAKEAVKALEVAIESGDEQLIRAALSEVEAA |
| | | | VHLAKALLAKPESPLVDEGFELLKLAAKTLAAY |
| | | | AEGEDVDKIALKLKAISAMAEALRLALAGDLER |
| | | | AARAAEEAVRYAIEAGDKELLRLAAEVAAYIAR |
| | | | LAEEAGLEEVARRAREAAERAREAAK |
| 7 | GLP1 | Threading | SPEEEARRAAREAERAAREAREAARRLGDEESV |
| | | | RVAERLEREARRAERERDLELARRVLRAAEALR |
| | | | LALEGELLAREQGDELGVVVARMITLAARDSAL |
| | | | GRGTPELARLLLRVARALLEGDLEEVVRSLAEI |
| | | | AKREIGTERALLAVEAIKLVALESIEEGDFETA |
| | | | ELAIEKLREIAEEFEGTEVAEKAREAIEEIEKK |
| | | | KREAE |
| 8 | GCG | RF Diffusion | SMEKLAEIMQEIIEAYQEVKDAFFKFIKAVHEG |
| | | (partial) | APEEELKKYLEKMKEALEKMKELLERLEKEAKK |
| | | | VIEENKDKKLELKVLLMLRLAYLLLKVSIELTK |
| | | | IAAEKLGDKELVEELEKESKEVEKKIKELEERI |
| | | | KKLLEEVDDEELKEAYKEVEEMEKEZEKFLEKM |
| | | | RKV |
| 9 | NPY | RF Diffusion | GMEERRKELLEKLKKLKEEVVELFRELAQALRD |
| | | (partial) | GASKERLEEIRERAEKLAEEAKKVAEELEKLAE |
| | | | GDAVLQLYLAEAYALEAAALTIEAVAAAELGAS |
| | | | KEELEKIKEKIEEALKKAEEAMKKALAEAKARG |
| | | | RERLVRLIEEARKEFEKLSKAIKELLEQV |
| 10 | PTH | RF Diffusion | MREKLEEMLEEFNEVIDELIEITKEDAPELEEL |
| | | | RERAEEAVENERLDELEEILDELEVIILEAMER |
| | | | DLSAAIEMTKAKNDKEKLKELLKQLEELEKRIK |
| | | | ELLERAKKRGNKKIIEKLEKLLKEVEKLKKEIE |
| | | | EYLK |
| 11 | Bim | RF Diffusion | EEERKEKREKVRAGLKRAIAELPAEVAARCLAL |
| | | | LDDASDEEFIEAVLEVLEAMREALVAMAREGRL |
| | | | DAVRRATSHINEVLVDAAELALEKGREYFRRLC |
| | | | LIVCDMMIELIRLEPEQTPELRRIRERLEEIRR |
| | | | RLE |
| 12 | PYY | RF | GLEEAEKLLEEIFANFEEIVELIKKNIGTERGK |
| | | Diffusion_fle | KLLKVFVATVDLILARLEQGADLAELAELVKEI |
| | | X | AELAKDEEGLEEAEKLVKELTAAR |

TABLE 2

| Protein design/ | Peptide | Target interacting | |
|---|---|---|---|
| Design method | target | residues | Applications |
| GCG_A04 | Glucagon | 6, 13, 83, 96, 97, 99, | (1) Diagnostic tool for glucagon- |
| (RFjoint) | | 100, 101, 143, 146, 147, | producing tumors, (2) assessment |
| SEQ ID NO: 3 | | 161, 162, 164 | of diabetic patients and other |
| GCG_C09 | | 16, 17, 20, 24, 28, 79, | carbohydrate metabolism |
| (RF Diffusion/ | | 82, 83, 86, 143, 144, | disorders, (3) diagnosis of |
| Partial | | 146, 147, 150, 157, 161 | glucagon deficiency in patients |
| diffusion) | | | with hypoglycemia, and (4) |
| SEQ ID NO: 8 | | | detection tool for diabetes and |
| | | | obesity research |
| GLP1_B04 | Glucagon-like | 57, 74, 78, 90, 107, | (1) Diagnostic tool for |
| (Sequence | peptide 1 | 111, 114, 116 | insulinomas, (2) assessment of |
| threading) | | | diabetic patients and other |
| SEQ ID NO: 7 | | | carbohydrate metabolism |
| | | | disorders, and (3) detection tool |
| | | | for diabetes and obesity research |
| SCT_A02 | Secretin | 79, 91, 153, 160 | (1) Diagnostic tool for pancreatic |
| (Sequence | | | insufficiency, and (2) detection of |
| threading) | | | gastrin-secretin tumors |
| SEQ ID NO: 5 | | | |
| GIP_A08 | Gastric | 80, 85, 86, 90, 91, | (1) Assessment of obese and |
| (Sequence | inhibitory | 101, 129, 139, 141, | diabetic patients, and (2) |
| threading) | peptide | 142 | detection tool for diabetes and |
| SEQ ID NO: 6 | | | obesity research |
| PTH_B10 | Parathyroid | 2, 12, 13, 15, 16, 19, | (1) Diagnostic tool for |
| (RFjoint) | hormone | 20, 23, 60, 70, 71, 74, 75, | calcium/phosphate disorders, (2) |
| SEQ ID NO: 2 | | 76, 77, 79, 122, 126, | assessment of patients with |
| | | 129, 130, 133 | hyperparathyroidism, (3) |
| PTH_D08 | | 7, 8, 15, 18, 53, 71 | parathyroid cancer, and (4) |
| (RF Diffusion/ | | | chronic kidney diseases-mineral |
| Partial | | | and bone disorder |
| diffusion) | | | |
| SEQ ID NO: 10 | | | |
| NPY_A9 | Neuropeptide Y | 16, 17, 18, 21, 26, 27, 28, | (1) Diagnostic tool for major |
| (RFjoint) | | 43, 53, 73, 93, 100, 103, | depression, (2) anxiety disorders, |
| SEQ ID NO: 4 | | 106, 135, 146, 150 | (3) Alzheimer's, (4) Parkinson, |
| NPY_C5 | | 24, 73, 133, 145, 150, | and (5) obesity |
| (RF Diffusion/ | | 151, 156, 157 | |
| Partial | | | |
| diffusion) | | | |
| SEQ ID NO: 9 | | | |
| PPY_A04 | Peptide YY | 17, 73, 88, 140, 150, | (1) Diagnostic tool for colorectal |
| (RF Diffusion_flex) | | 153, 156 | cancer, (2) heart failure, (3) |
| SEQ ID NO: 12 | | | assessment of diabetic patients, |
| | | | and (4) detection tool for diabetes |
| | | | and obesity research |
| BIM_E09 | BIM BH3 | 69, 75, 79, 80, 114, | (1) Detection tool for apoptosis |
| (RF Diffusion) | peptide | 121 | research, (2) measurement of |
| SEQ ID NO: 11 | | | cellular priming levels, and (3) |
| | | | development of Mcl-1 targeting |
| | | | therapeutics |

In one embodiment, the polypeptides comprise an amino acid sequence at least 50%, 55%, 600%, 65%, 700%, 750%, 80%, 850%, 900%, 91%, 92%, 93%, 94%, 950%, 960%, 97%, 980%, 99%, or 100% identical to the amino acid sequence selected from the group consisting of SEQ ID NO: 1, 2, and 10, wherein the polypeptide binds to parathyroid hormone (PTH). As described in Table 2, such polypeptides may be used, for example, for (1) diagnosis of calcium/phosphate disorders. (2) assessment of patients with hyperparathyroidism. (3) detection of parathyroid cancer, and (4) detection of chronic kidney diseases-mineral and bone disorder. In one such embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO: 2, wherein at least 1, 2, 3, 4, 5, 6, 7, 8, 9, 10, 11, 12, 13, 14, 15, 16, 17, 18, 19, 20 or all of residues 2, 12, 13, 15, 16, 19, 20, 23, 60, 70, 71, 74, 75, 76, 77, 79, 122, 126, 129, 130, and 133 are identical relative to SEQ ID NO:2. In another such embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:10, wherein at least 1, 2, 3, 4, 5, or all of residues 7, 8, 15, 18, 53, and 71 are identical relative to SEQ ID NO:10.

In another embodiment, the polypeptides may comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:3 and 8, wherein the polypeptide binds to glucagon (GCG). As described in Table 2, such polypeptides may be used, for example, for (1) diagnosis of glucagon-producing tumors, (2) assessment of diabetic patients and other carbohydrate metabolism disorders, (3) diagnosis of glucagon deficiency in patients with hypoglycemia, and (4) detection tool for diabetes and obesity research. In one such embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:3, wherein at least 1, 2, 3, 4, 5, 6, or more, or all of residues 6, 13, 83, 96, 97, 99, 100, 101, 143, 146, 147, 161, 162, and 164 are identical relative to SEQ ID NO:3. In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:8, wherein at least 1, 2, 3, 4, 5, 6, 7, 8, 9, 10, 11, 12, 13, 14, 15, or all of residues 16, 17, 20, 24, 28, 79, 82, 83, 86, 143, 144, 146, 147, 150, 157, and 161 are identical relative to SEQ ID NO:8.

In a further embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:4 and 9, wherein the polypeptide binds to neuropeptide Y (NPY). As described in Table 2, such polypeptides may be used, for example, for (1) diagnosis of major depression, (2) diagnosing anxiety disorders, (3) diagnosing Alzheimer's Disease, (4) diagnosing Parkinson's Disease, and (5) treating obesity. In one such embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:4, wherein at least 1, 2, 3, 4, 5, 6, 7, 8, 9, 10, 11, 12, 13, 14, 15, 16, or all of residues 16, 17, 18, 21, 26, 27, 28, 43, 53, 73, 93, 100, 103, 106, 135, 146, and 150 are identical relative to SEQ ID NO:4. In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:9, wherein at least 1, 2, 3, 4, 5, 6, 7, or all of residues 24, 73, 133, 145, 150, 151, 156, and 157 are identical relative to SEQ ID NO:9.

In one embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:5, wherein the polypeptide binds to secretin (SCT). As described in Table 2, such polypeptides may be used, for example, for (1) diagnosing pancreatic insufficiency, and (2) detecting gastrin-secretin tumors. In one such embodiment, at least 1, 2, 3, or all of residues 79, 91, 153, and 160 are identical relative to SEQ ID NO:5.

In another embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:6, wherein the polypeptide binds to gastric inhibitory peptide (GIP). As described in Table 2, such polypeptides may be used, for example, for (1) assessment of obese and diabetic patients, and (2) detecting diabetes. In one such embodiment, at least 1, 2, 3, 4, 5, 6, 7, 8, 9, or all of residues 80, 85, 86, 90, 91, 101, 129, 139, 141, and 142 are identical relative to SEQ ID NO:6.

In a further embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7, wherein the polypeptide binds to glucagon-like peptide I (GLP1). As described in Table 2, such polypeptides may be used, for example, for (1) diagnosing insulinomas, (2) assessment of diabetic patients and other carbohydrate metabolism disorders, and (3) detecting diabetes. In one such embodiment, at least 1, 2, 3, 4, 5, 6, 7, or all of residues 57, 74, 78, 90, 107, 111, 114, and 116 are identical relative to SEQ ID NO:7.

In one embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:11, wherein the polypeptide binds to BIM BH3 peptide (Bim). As described in Table 2, such polypeptides may be used, for example, (1) as a detection tool for apoptosis research, (2) in measurement of cellular priming levels, and (3) in developing Mcl-1 targeting therapeutics. In one such embodiment, at least 1, 2, 3, 4, 5, or all of residues 69, 75, 79, 80, 114, and 121 are identical relative to SEQ ID NO:11.

In a further embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:12, wherein the polypeptide binds to peptide YY (PYY). As described in Table 2, such polypeptides may be used, for example, for (1) diagnosing colorectal cancer, (2) diagnosing heart failure, (3) assessing diabetic patients, and (4) detecting diabetes. In one such embodiment, at least 1, 2, 3, 4, 5, 6, or more, or all of residues 17, 73, 88, 140, 150, 153, and 156 are identical relative to SEQ ID NO:12.

In one embodiment of any of these embodiments, substitutions relative to the reference sequence are conservative amino acid substitutions. Such conservative amino acid substitutions involve replacing a residue by a residue having similar physiochemical characteristics, e.g., substituting one aliphatic residue for another (such as Ile, Val, Leu, or Ala for one another), or substitution of one polar residue for another (such as between Lys and Arg; Glu and Asp: or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q). (3) acidic: Asp (D), Glu (E), (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe.

In another embodiment, the polypeptide binds its target with nanomolar or picomolar affinity in fluorescence polarization binding assays conducted using peptide binders and fluorescently labeled peptide targets m a buffer solution. The peptide binders are serially diluted and incubated with a constant concentration of the target peptides. After incubation, fluorescence polarization is measured at specific wavelengths to determine the binding affinities, with the data analyzed using curve fitting.

In a further embodiment, the disclosure provides fusion proteins, comprising:
(a) the polypeptide of any embodiment herein; and
(b) one or more functional domains.

In various non-limiting embodiments, the functional domain may comprise, for example, a targeting domain, a detectable domain, a scaffold domain, a secretion signal, an Fc domain, or a therapeutic peptide domain. These domains provided added functionality to the polypeptides of the disclosure, including but not limited to permitting detection and increasing serum half-life of the polypeptides.

In another aspect the disclosure provides nucleic acids encoding the polypeptide or fusion protein of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded peptide or chimeric molecular construct, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptide or fusion protein of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptide, fusion protein nucleic acid or expression vector (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another aspect, the disclosure provides pharmaceutical compositions, comprising:
(a) the polypeptide, fusion protein, nucleic acid, expression vector, and/or host cell of any embodiment herein; and
(b) a pharmaceutically acceptable carrier.

The pharmaceutical compositions may be used, for example, for any of the various methods of using the polypeptides for treating or diagnosing disease as described herein. The compositions may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity-adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

In a further aspect, the disclosure provides methods, comprising contacting a biological sample with the polypeptide or fusion protein of any embodiment, and detecting binding of the polypeptide or fusion protein with target present in the biological sample, wherein presence of the biological sample correlates with a disease state. Table 2 provides various exemplary diagnostic uses of the polypeptides and fusion proteins of the disclosure. For example, in embodiments where the polypeptide or fusion proteins of claims 2-4 (PTH binders) are used, the methods can be used to assess a subject for disorders associated with PTH, including (1) calcium/phosphate disorders, (2) assessment of patients with hyperparathyroidism, (3) parathyroid cancer, and (4) chronic kidney diseases-mineral and bone disorder. Those of skill in the art will understand other diagnostic/prognostic methods that can be carried out using the polypeptides and fusion proteins of the disclosure as detailed in Table 2.

Any biological sample may be used as appropriate for an intended purpose, including but not limited to blood, saliva, urine, sweat, tears, plasma, serum, milk, spinal fluid, lymph fluid, secretions from the respiratory tract, secretions from the intestinal tract, and secretions from the genitourinary tract. The biological sample may be obtained from any host, including a mammal, such as a human.

Such detecting can be carried out by any suitable means, including those described in the examples that follow.

### EXAMPLES

### Abstract

We describe parametric generation and deep learning-based methods for designing proteins to address this challenge. We show that by extending RF Diffusion to enable binder design to flexible targets, and to refining input structure models by successive noising and denoising (partial diffusion), picomolar affinity binders can be generated to helical peptide targets both by refining designs generated with other methods, or completely de novo starting from random noise distributions; to our knowledge these are the highest affinity designed binding proteins against any protein or small molecule target generated directly by computation without any experimental optimization. The RF Diffusion designs enable the enrichment of parathyroid hormone or other bioactive peptides in human plasma and subsequent detection by mass spectrometry, and bioluminescence-based protein biosensors.

### Main

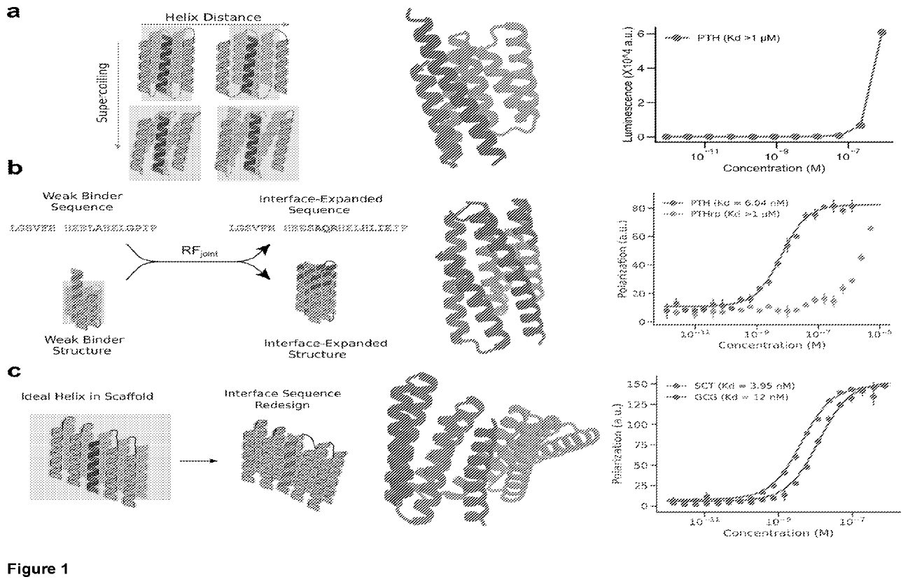

Peptide hormones, such as parathyroid hormone (PTH), neuropeptide Y (NPY), glucagon (GCG), and secretin (SCT), which adopt alpha helical structures upon binding their receptors, play key roles in human biology and are well established biomarkers in clinical care and biomedical research (FIG. 1a). There is considerable interest in their sensitive and specific quantification, which currently relies on antibodies that require substantial resources to generate, can be difficult to produce with high affinity, and often have less-than-desirable stability and reproducibility; the loop-mediated interaction surfaces of antibodies are not particularly well suited to high specificity binding of extended helical peptides—almost all anti-peptide antibodies bind their targets in non-helical conformations. The design of proteins that bind helical peptides with high affinity and specificity remains an outstanding challenge. Design of peptide-binding proteins is challenging for two reasons. First, proteins designed to bind folded proteins, have shapes suitable for binding rigid concave targets, but not for cradling extended peptides. Second, peptides have fewer residues to interact with, and are often partially or entirely unstructured in isolation; as a result, there can be an entropic cost of structuring the peptide into a specific conformation, which compromises the favorable free energy of association.

### Design of Helical Peptide Binding Scaffolds

We set out to develop general methods for designing proteins that bind peptides in helical conformations. To fully leverage recent advances in protein design, we explored both parametric and deep learning-based approaches. For parametric generation, we reasoned that helical bundle scaffolds with an open groove for a helical peptide could provide a general solution to the helical peptide binding problem: the extended interaction surface between the full length of the helical peptide target and the contacting helices on the designed scaffold could enable the design of high affinity and specificity binding, while the helices flanking the groove limit self-association of the recessed hydrophobic surfaces. In parallel, we reasoned that deep learning methods, which do not pre-specify scaffold geometries, could permit the exploration of different potential solutions to peptide binding.

### Parametric Design of Groove Scaffolds

We began by exploring parametric methods for generating backbones with overall “groove” shapes. Using the Crick parameterization of alpha-helical coiled coils²⁵, we devised a method to sample scaffolds consisting of a three helix groove supported by two buttressing helices (FIG. 1b, see Methods). We assembled a library of these scaffolds sampling a range of supercoiling and helix-helix spacings to accommodate a variety of helical peptide targets. We then used this library to design binders to PTH, GCG, and NPY, and screened 12 designs for each target using a nanoBiT split luciferase binding assay. Many of the designs bound their targets (3/12, 4/12, and 8/12 to PTH. GCG, and NPY) but with only micromolar affinities (FIG. 1b). These results suggest that groove-shaped scaffolds can be designed to bind helical peptides, but also that design method improvement was necessary to achieve high-affinity binding.

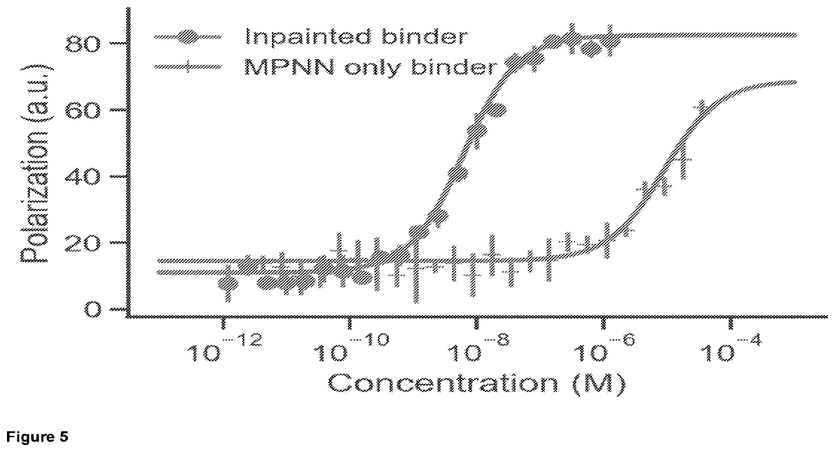

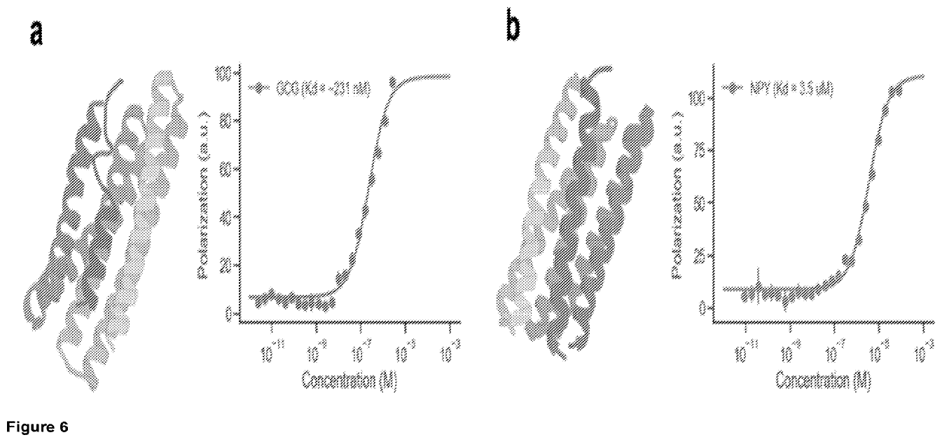

We next explored using RoseTTAFold™ inpainting (RFjoint)²⁶, a model that can jointly design protein sequences and structures, along with ProteinMPNN²⁷, an improved sequence design method, to improve the modest affinity of our tightest parametrically designed PTH binder (FIG. 1c, left). We used RF joint inpainting to extend the binder interfaces and ProteinMPNN to redesign the sequences, reasoning that the combination of these two methods could lead to more favorable interactions with the peptide. Out of 192 designs tested, 44 showed binding against PTH in initial yeast display screening. Following size exclusion chromatography (SEC), the best binder was found to bind with 6.1 nM affinity to PTH using fluorescence polarization (FP). Binding was quite specific: very little binding was observed to PTH related peptide (PTHrp), a related peptide sequence with 34% sequence identity which binds the same receptor as PTH²⁸ (FIG. 1c, right). Overall, the affinity of the starting PTH binder was improved by approximately three orders of magnitude, and the computational model of the highest-affinity binder had 19% greater surface area contacting the target peptide (in this case, the structural extension proved critical to the improvement in binding affinity; sequence redesign with ProteinMPNN of the original binding interface did not measurably increase affinity as shown in FIG. 5). We used the same design strategy to generate higher affinity binders for NPY and GCG. Using weak parametric binders as a starting point, we extended their binding interfaces and redesigned their sequences to generate a 231 nM affinity binder for GCG and a 3.5 μM binder for NPY after screening 96 designs (FIG. 6a, 6b).

Design of Helical Peptide Binders with Sequence Threading

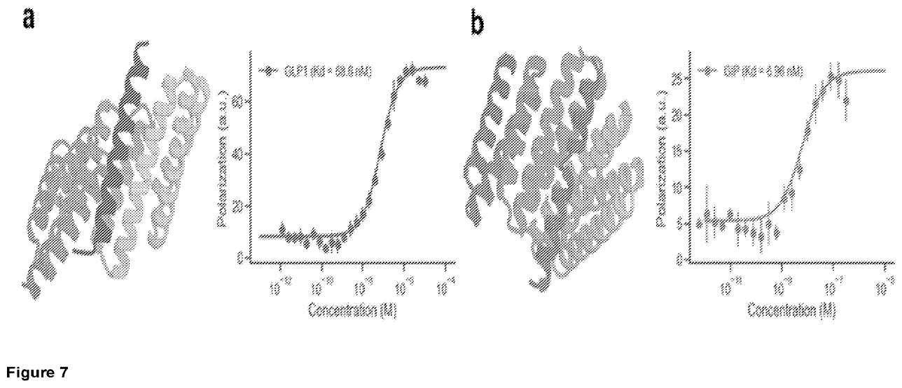

As an alternative to de novo parametric design of scaffolds that contain grooves, we explored the threading of helical peptides of interest onto already existing designed scaffolds with interfaces that make extensive interactions with helical peptides²⁹ (FIG. 1d, left). We started from a library of scaffolds that contained single helices bound by pseudorepetitive helical scaffolds³⁰. We then threaded sequences of peptides of interest onto the bound single helix and filtered to maximize interfacial hydrophobic interactions of the target sequence to the binder scaffold^{20,29}. The binders were then redesigned in the presence of the threaded target sequence with ProteinMPNN²⁷ and the complex was predicted with Alphafold³¹ (AF2; with initial guess⁸) and filtered on AF2 and Rosetta™ metrics. Initial screening using yeast surface display identified 4/66 binders, which were expressed in E. coli. After purification, all 4 of the designs were found to bind with sub-micromolar affinity using FP, with the highest-affinity design binding with an affinity of 2.7 nM for SCT (FIG. 1d, right), we also made designs with Kd<100 nM to Glucagon-like peptide 1 (GLP1) and Gastric inhibitory polypeptide (GIP, FIG. 7a, 7b). The binding specificity of the SCT design was assessed with FP by measuring affinity for GCG, a related hormone with 44% sequence identity to SCT^{4,32}. We found that the tightest SCT binder was only 4 fold selective for SCT over GCG, which suggested additional design strategies might be necessary to increase the quality of the binding interface and to achieve high-specificity binding (FIG. 1d, right).

### Designing Peptide Binders by Hallucination

We next explored the use of deep learning hallucination methods to generate helical peptide binders completely de novo, with no pre-specification of the desired binder geometry (from peptide sequence alone) (FIG. 1e, left). Hallucination or “activation maximization” approaches start from a network that predicts protein structure from sequence, and carry out an optimization in sequence space for sequences which fold to structures with desired properties. This approach has been used to generate novel monomers³³, functional-site scaffolds²⁶ and cyclic oligomers³⁴. Hallucination using AlphaFold2 (AF2) or RosettaFold™ has a number of attractive features for peptide binder design. First, neither the binder nor the peptide structure needs to be specified during the design process, enabling the design of binders to peptides in different conformations (this is useful given the unstructured nature of many peptides in solution; disordered peptides have been observed to bind in different conformations to different binding partners²¹). Second, metrics such as the predicted alignment error (pAE) have been demonstrated to correlate well with protein binding⁸, permitting the direct optimization of the desired objective, albeit with the possible hazard of generating adversarial examples¹⁴.

Peptide Binder Design with RF Diffusion

We next explored further developing and applying the RoseTTAFold™-based denoising diffusion method RF Diffusion introduced in the accompanying paper (Watson et al.) RF Diffusion is much more compute efficient than hallucination, as it is trained to directly generate a diversity of solutions to specific design challenges starting from random 3D distributions of residues that are progressively denoised. We sought to extend the RF Diffusion approach for general protein structure refinement and binder optimization (by sampling related conformations around a provided input structure) and for fully de novo design starting from a completely random noise distribution and either the target amino acid sequence alone or the sequence and structure of the target.

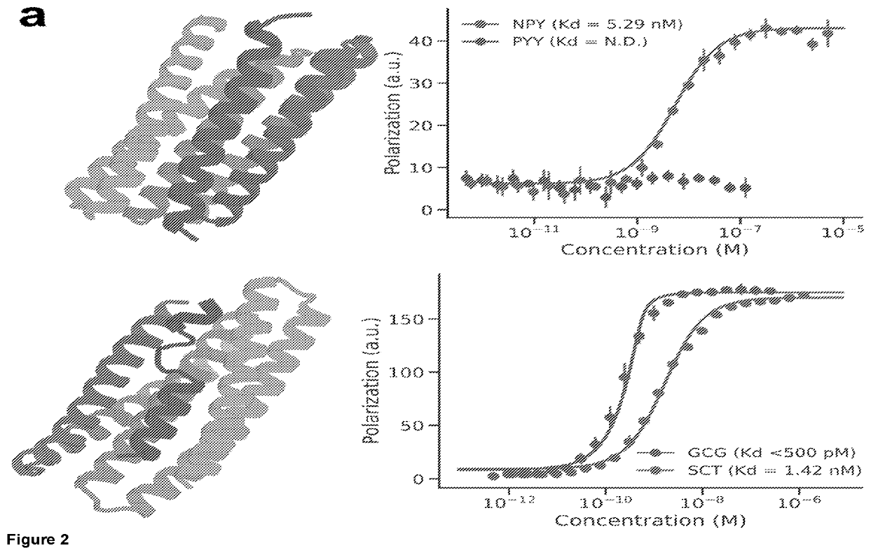

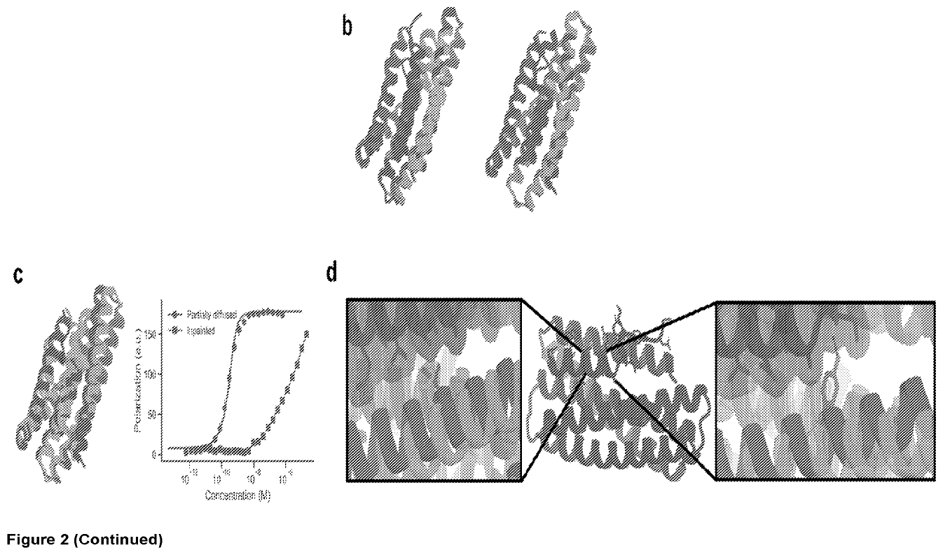

A long standing challenge in protein design is to increase the activity of an input native protein or designed protein by exploring the space of plausible closely related conformations for those with predicted higher activity.³⁷ This is difficult for traditional design methods as extensive full atom calculations are needed for each sample around a starting structure (using molecular dynamics simulation or Rosetta™ full atom relaxation methods), and it is not straightforward to optimize for higher binding affinity without detailed modeling of the binder-target sidechain interactions. We reasoned that, in contrast, RF Diffusion might be able to rapidly generate plausible backbones in the vicinity of a target structure, increasing the extent and quality of interaction with the target guided by the extensive knowledge of protein structure inherent in RoseTTAfold™. During the reverse diffusion (generative) process, RF Diffusion takes random Gaussian noise as input, and iteratively refines this to a novel protein structure over many (“T”) steps (typically 200). Partly through this denoising process, the evolving structure no longer resembles “pure noise”, instead resembling a “noisy” version of the final structure. We reasoned that ensembles of structure with varying extents of deviation from an input structure could be generated by partially noising to different extents (for example, timestep 70), and then denoising to a similar, but not identical final structure (FIG. 2a).

We implemented this “partial diffusion” approach (see code availability section), and sought first to assess the extent to which protein structures could be resampled and refined with partial diffusion. As expected, partial diffusion allowed diversification of a starting protein fold, and the magnitude of this diversity could be tuned by varying how many steps of noise were initially added to a starting structure (FIG. 2a). We next explored the ability of partial diffusion to regularize protein backbones using as a metric AF2 structure prediction starting from a single sequence; for many native proteins this fails due to irregularities in both sequence and structure. We found that RF Diffusion improves the “designability” of protein backbones, ProteinMPNN sequence design on partially diffused native backbones (with high similarity to the native fold) improved structure recapitulation by AF2 relative to both the native sequence and ProteinMPNN sequences generated from the native backbone. We found further in tests on the well-studied colicin-immunity protein system that partial diffusion can efficiently sample the small changes in protein backbone geometry that accompany specificity changes within protein families. Thus, partial diffusion enables protein backbone resampling and refinement, the extent of which can be tuned by varying the amount of noise added, and can considerably increase the designability of input protein models.

As a first experimental test of partial diffusion, we started from our parametrically-designed, inpainted binders to GCG (with 231 nM Kd) and NPY (with 3.5 μM affinity) (FIG. 6). Following partial noising and denoising, we identified designs that in silico, had significantly improved computational metrics compared to the starting design. We used an auxiliary potential during the denoising trajectory⁹ which minimized the radius of gyration (see accompanying code) of the protein-peptide complex to promote compact binding to the peptide. The diversity compared to the starting design could be readily tuned by varying the time point to which the starting design was noised (FIG. 2a). Initial screening on yeast display revealed quite high binding success rates, with 25/96 designs binding GCG, and 20/96 binding NPY at 10 nM peptide concentration. The highest affinity designs were expressed in E. coli, purified, and their binding affinities were determined using FP. The highest-affinity binders were found to bind at 5.6 nM to NPY (FIG. 2b, left), and bound GCG with subnanomolar affinity (FIG. 2b, right). The designs were quite specific: the GCG binders bound 10 times less tightly to SCT, which was chosen due to its high similarity to GCG. Impressively, the NPY binder did not show any cross-reactivity to peptide YY (PYY), which is a member of the NPY/pancreatic polypeptide family³⁸ with 63% sequence identity.

To gain insight into the structural rearrangements generated by partial diffusion that contribute to the affinity increases, we solved the structures of the original inpainted GCG binder and the partially diffused higher affinity version. Subtle structural changes at the designed interface between the original inpainted design model (FIG. 2c, left) and the partially diffused model (FIG. 2c, right), are nearly perfectly recapitulated in the corresponding crystal structures (FIG. 2c, 1.95 Å, 0.72 Å RMSD for the inpainted design, 1.81 Å, 0.6 Å RMSD for the partially diffused design). A 0.4 Å shift towards the target in the binder backbone enables an Ile to fit into a pocket previously occupied by a poorly packed Phe sidechain (FIG. 2e, left, middle). Direct substitution of the Phe with the Ile in the original design model prior to partial diffusion does not fill the pocket (FIG. 2e, right).

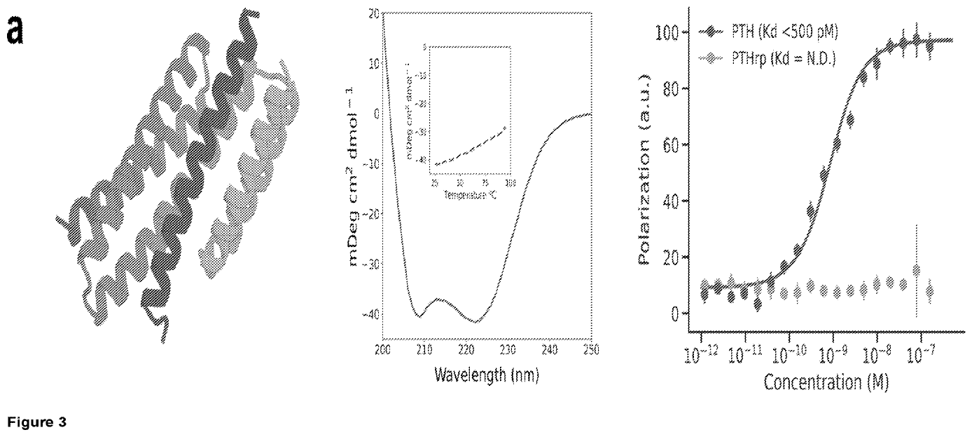

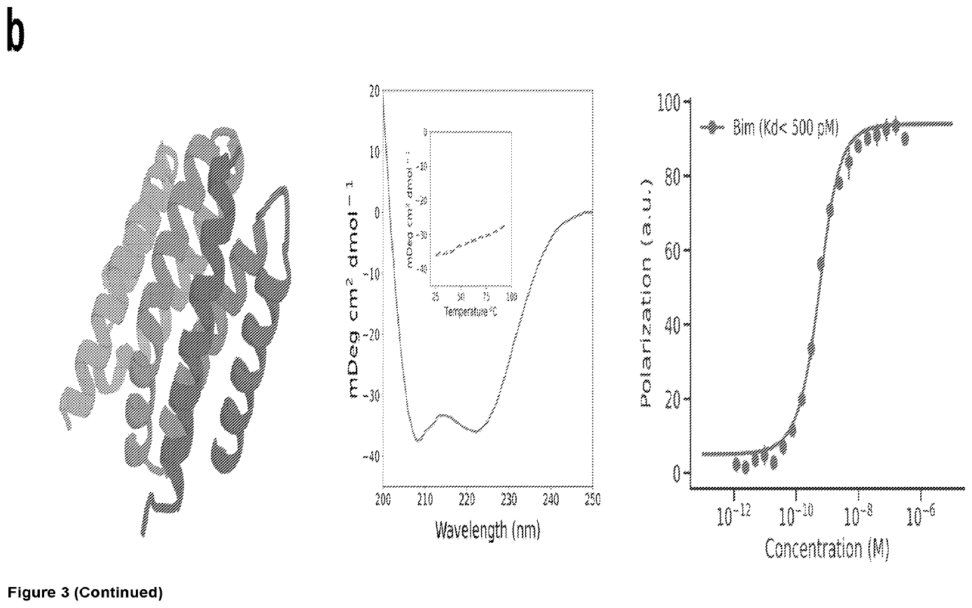

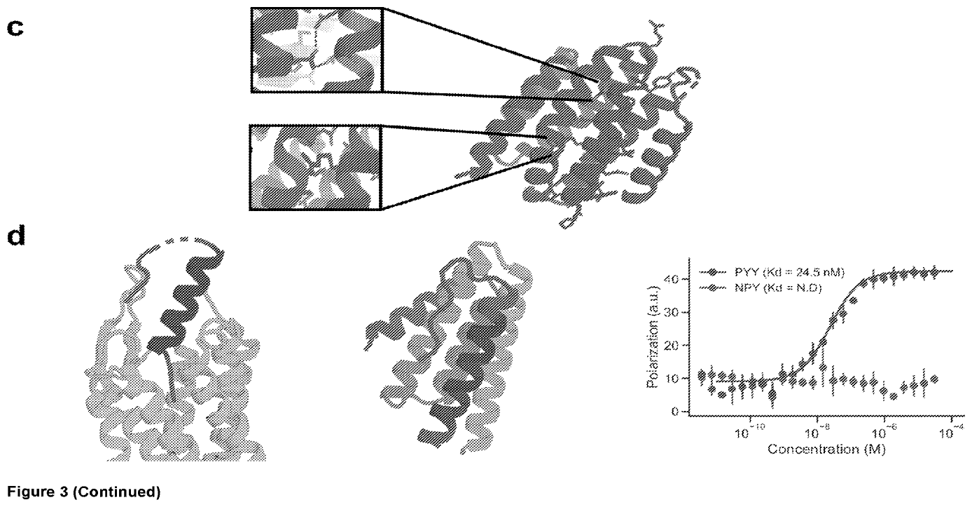

Inspired by this success at optimizing binders with RF Diffusion, we next tested its ability to design binders completely de novo through unconditional binder design. We first used the fixed target structure approach of Watson et al, and provided RF Diffusion with the sequence and structures of the two peptides in helical conformations, leaving the topology of the binding protein and the binding mode completely unspecified (FIG. 3a). From this minimal starting information, RF Diffusion generated designs predicted by AF2 to fold and bind to the targets with high in silico success rates. A representative design trajectory is shown for PTH in Supplemental Video 1; starting from a random distribution of residues surrounding the PTH peptide in a helical conformation, in sequential denoising steps the residue shifts to surround the peptide and progressively organize itself into a folded structure which cradles the peptide along its entire surface.

We obtained synthetic genes encoding 96 designs for each target. Using yeast surface display, we found that 56 of the 96 designs bound to PTH at 10 nM peptide concentration. The highest affinity design again bound too tightly for accurate Kd estimation; instead FP data provides an approximate upper bound for the Kd<500 pM (FIG. 3b, right). Binding was also highly specific; no binding was observed to the related PTHrp (FIG. 3b, right) For Bim, we found 25/96 of the designs bound by yeast surface display with sub-micromolar affinities. The highest affinity design was found to bind with a Kd<500 pM (FIG. 3c, right). Circular dichroism temperature melts indicate that both binders are stable at 95° C. (FIG. 3b, middle, 3c, middle). The diffused from scratch binders again had considerable structural similarity to our starting groove binding concept. We were able to solve the X-ray crystallographic structure of the Bim binder, and found that it matched the design model with atomic accuracy (3.0 Å, 0.57 Å RMSD, FIG. 3d, middle). A kinked helix on the binder designed adjacent to the interface, likely to accommodate Arg 13 of the Bim peptide, is well-recapitulated in the structure, and a cross-interface hydrogen bond network forms using Thr 73 and Asn 77 of the binder to allow satisfactory burial of Asn 20 of Bim at the otherwise hydrophobic interface.

We next sought to generalize RF Diffusion to enable binding to flexible targets from a specification of the target sequence alone. We finetuned RF Diffusion by training on two chain systems from the PDB, noising the structure on one and providing only the sequence on the second We found that the fine-tuned version could readily design folded structures around a variety of peptides given only sequence information. We used this approach to design binders to PYY (FIG. 3e), which in the cryoEM structure with the Neuropeptide Y Y2 receptor adopts a partially helical structure³⁹. Starting from only the amino acid sequence of PYY, RF Diffusion_flex generated solutions with the peptide in a range of conformations. Experimental characterization of a design with the peptide adopting a different conformation than the experimental structure showed it bound with 24.5 nM affinity (FIG. 3e, right; we explored using shorter chain lengths in these calculations, resulting in smaller designs, which likely accounts for the lower affinity than in the fixed structure case above).

### Comparison of Human and Machine Based Problem Solving

The deep learning methods largely converged on the overall solution to the helical peptide binding design problem-groove shaped scaffolds with helices lining the binding site that the human designers chose in the initial Rosetta™ parametric approaches. The increased affinity of the deep learning designs likely derives at least in part from higher shape complementarity resulting from direct building of the scaffold to match the peptide shape, the average contact molecular surface for the partially diffused GCG binders and NPY increased by 33% and 29% respectively compared to the starting models, and the Rosetta™ ddG improved by 29% and 21%. The ability of RF Diffusion de novo design to “build to fit” provides a general route to creating high shape complementary binders to a wide range of target structures, and as noted above, partial diffusion provides a very general route to increasing affinity by making small backbone adjustments to enable placement of more space filling sidechains.

### Design of Protein Biosensors for PTH Detection

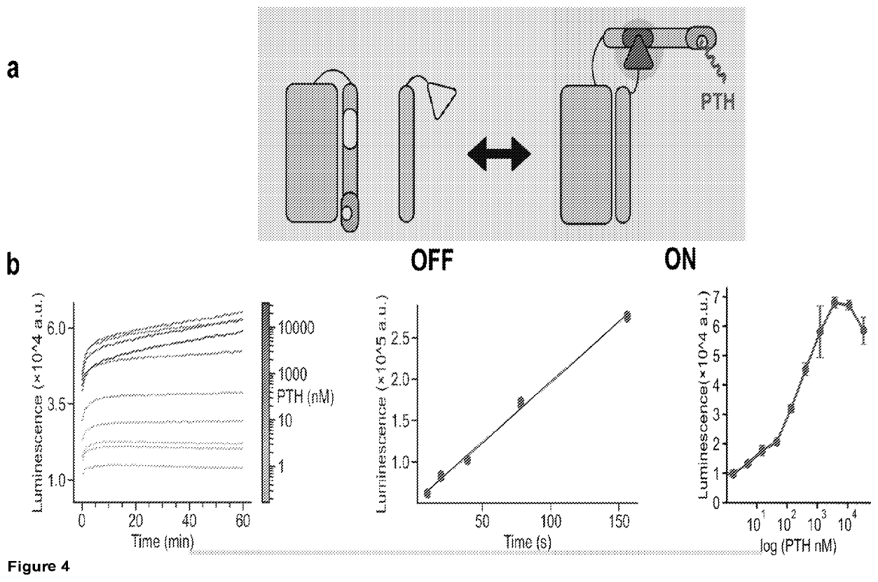

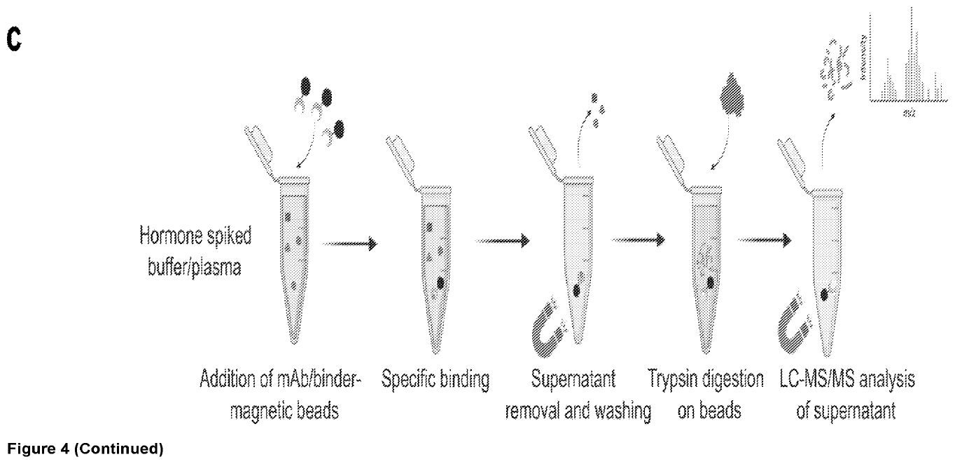

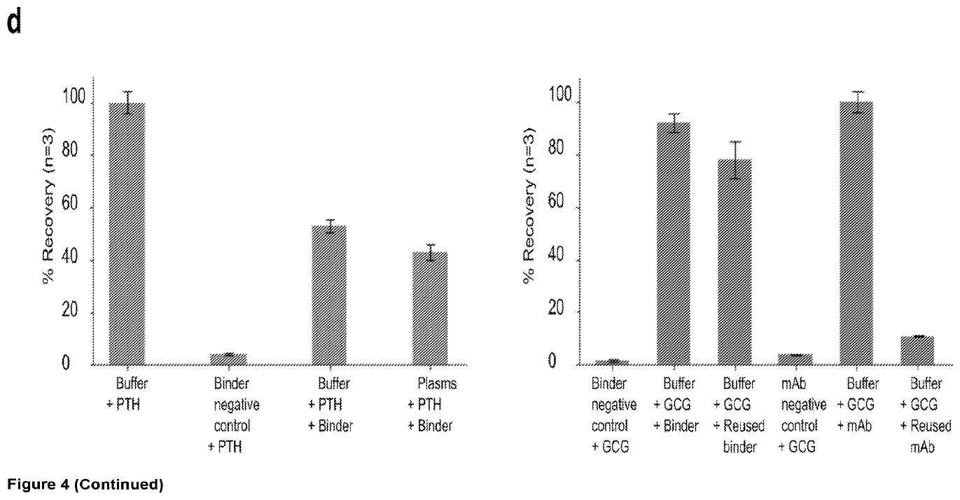

Given our success in generating de novo binders to clinically-relevant helical peptides, we next sought to test their use as detection tools for use in diagnostic assays. Compared to immunosensors, de novo protein-based biosensors offer a more robust platform with high stability and tunability for diagnostics^{40, 41}. To design PTH biosensors, we grafted the 6.1 nM PTH binder into the lucCage system⁴² (FIG. 4a), screened 8 designs for their luminescence response in the presence of PTH, and identified a sensitive lucCagePTH biosensor (LOD=10 nM) with ~21-fold luminescence activation in the presence of PTH (FIG. 4b).

Enriching Peptide Targets from a Complex Mixture

We explored the use of our picomolar affinity RF Diffusion generated binder to PTH as a capture reagent in immunoaffinity enrichment coupled with liquid chromatography-tandem mass spectrometry (LC-MS/MS), a powerful platform for detecting low-abundance protein biomarkers in human serum. We evaluated the RF Diffusion binder in an LC-MS/MS assay for PTH in scrum⁴³. PTH enrichment was quantified based on the analysis of the N-terminal peptide of a tryptic digestion of PTH in human plasma^{44,45}. (see Methods). We found that the designed binder enabled capture of PTH from spiked buffer and spiked human plasma with recoveries of 53% and 43%, respectively (FIG. 4d). The very high thermal stability of the designed binders (FIG. 3b middle, 3c, middle) suggests that capture reagents based on them could have much longer shelf lives than antibodies, and be amenable to harsher washing conditions enabling re-use of binder conjugated beads.

### DISCUSSION

Antibodies have served as the industry standard for affinity reagents for many years, but their use is often hampered by variable specificity and stability. For binding helical peptides, the computationally designed helical scaffolds described in this paper have a number of structural and biochemical advantages. First, the extensive burial of the full length of an extended helix is difficult to accomplish with antibody loops, but very natural with matching extended alpha helices in groove shape scaffolds. Second, designed scaffolds are more amenable to incorporation into sensors as illustrated by the LucCage Pil sensor. Third, they are more stable, can be produced much less expensively, and could be more easily incorporated into affinity matrices for enrichment of peptide hormones from human serum. Fourth, peptide binders can achieve high affinity and specificity purely through computational methods, eliminating the need to use animals, which often mount weak responses to highly conserved bioactive molecules. Our MS based detection of peptides present at very low abundance in sera following enrichment using the designed binders could provide a general route forward for serological detection of a wide range of disease associated peptide biomarkers.

Our results highlight the emergence of powerful new deep learning methods for protein design. The inpainting and RF Diffusion methods were both able to improve on initial Rosetta™ designs, and the hallucination approach generated high affinity binders without requiring prespecification of the bound structures. Most impressively, the RF Diffusion method rapidly generated very tight (picomolar Kds) affinity and specific binders to multiple helical peptides. Here we demonstrate further that RF Diffusion can be used to improve starting designs by partial noising and denoising, and can generate binders to peptides starting from no information other than the target structure or sequence. To our knowledge, the Bim and PTH binding proteins diffused starting from random noise are the highest affinity binders to any target (protein, peptide, or small molecule) achieved directly by computational design with no experimental optimization. We expect both the de novo peptide binder design capability and the ability to resample around initial designs (before or after experimental characterization) to be broadly applicable.

TABLE 3
Alphafold metrics for partially and fully diffused binders.

| | GCG | NPY | PTH | Bim |
|---|---|---|---|---|
| | Binder | Binder | Binder | Binder |
| RMSD AF2 vs | 0.62 Å | 0.61 Å | 0.78 Å | 0.80 Å |
| Design | | | | |
| AF2 interaction | 9.25 | 8.29 | 4.40 | 4.50 |
| pAE | | | | |
| AF2 pLDDT for | 95.52 | 93.41 | 94.3 | 96.6 |
| binder | | | | |

TABLE 4
Crystallographic data collection and refinement

| | GCG_partdiff | GCG_inpaint | Bim_fulldiff | PTH |
|---|---|---|---|---|
| | (8GJI) | (8GJG) | (8T5E) | (8T5F) |
| Data Collection | | | | |
| Space group | P 2₁ 2₁ 2₁ | P 2₁ | P 2₁ 2₁ 2₁ | P 4 2₁ 2 |
| Cell dimensions | | | | |
| a, b, c (Å) | 31.26, 50.92, 91.92 | 37.79, 45.90, 63.60 | 25.79, 66.67, 74.30 | 91.32, 91.32, 37.73 |
| α, β, γ (°) | 90, 90, 90 | 90, 97.31, 90 | 90, 90, 90 | 90, 90, 90 |
| Resolution (Å) | 91.93-1.81 | 63.09-1.95 | 74.30-3.00 | 40.84-1.99 |
| | (1.88-1.81) | (2.00-1.95) | (3.18-3.00) | (2.04-1.99) |
| R_{merge} | 0.099 (1.581) | 0.073 (2.001) | 0.064 (0.173) | 0.120 (2.069) |
| Rₚᵢₘ | 0.053 (0.761) | 0.033 (0.884) | 0.028 (0.073) | 0.033 (0.548) |
| I/σ(I) | 8.3 (0.90) | 10.10 (0.60) | 17.8 (8.8) | 21.8 (1.3) |
| CC_{1/2} | 0.993 (0.459) | 0.998 (0.461) | 0.999 (0.985) | 0.999 (0.545) |
| Completeness (%) | 99.60 (99.40) | 98.50 (96.30) | 99.9 (100) | 98.6 (94.6) |
| Redundancy | 6.2 (6.0) | 6.7 (6.8) | 7.2 (7.9) | 15.1 (15.2) |
| Refinement | | | | |
| Resolution (Å) | 45.96-1.81 | 63.09-1.95 | 49.62-3.00 | 40.84-1.99 |
| | (1.88-1.81) | (2.00-1.95) | (49.62-3.00) | (2.19-1.99) |
| R_{work}/R_{free} | 0.2080 (0.3752)/ | 0.2087 (0.4205)/ | 0.2398 (0.2398)/ | 0.2201 (0.2506)/ |
| | 0.2552 (0.4485) | 0.2488 (0.4445) | 0.2617 (0.2617) | 0.2494 (0.3372) |
| No. atoms | | | | |
| Protein | 1579 | 1539 | 1244 | 853 |
| Water | 24 | 26 | 0 | 26 |
| Ramachandran | 98.38/1.62/0.00 | 100.00/0.00/0.00 | 95.21/4.79/0.00 | 100/0.00/0.00 |
| Favored/allowed | | | | |
| Outlier (%) | | | | |
| R.m.s. deviations | | | | |
| Bond lengths (Å) | 0.012 | 0.002 | 0.003 | 0.010 |
| Bond angles (°) | 1.12 | 0.440 | 0.500 | 1.04 |
| B_{factors} (Å²) | | | | |
| Protein | 45.14 | 68.55 | 77.56 | 61.14 |
| Water | 47.64 | 69.57 | n/a | 62.39 |

### REFERENCES

1. Pioszak, A. A. & Xu, H. E. Molecular recognition of parathyroid hormone by its G protein-coupled receptor. Proc. Natl. Acad. Sci. U.S.A 105, 5034-5039 (2008).
2. Park, C. et al. Structural basis of neuropeptide Y signaling through Y1 receptor. Nat. Commun. 13, 853 (2022).
3. Sasaki, K., Dockerill, S., Adamiak, D. A., Tickle, I. J. & Blundell, T. X-ray analysis of glucagon and its relationship to receptor binding. Nature 257, 751-757 (1975).
4. Fukuhara, S. et al. Structure of the human secretin receptor coupled to an engineered heterotrimeric G protein. Biochem. Biophys. Res. Commun. 533, 861-866 (2020).
5. Wewer Albrechtsen, N. J., Kuhre, R. E., Pedersen, J., Knop, F. K. & Holst, J. J. The biology of glucagon and the consequences of hyperglucagonemia. Biomark. Med. 10, 1141-1151 (2016).
6. Khan, M., Jose, A. & Sharma. S. Physiology, Parathyroid Hormone. (StatPearls Publishing, 2022).
7. Cao, L. et al De novo design of picomolar SARS-CoV-2 miniprotein inhibitors. Science 370, 426-431 (2020).
8. Bennett, N. R. et al. Improving de novo protein binder design with deep learning. Nat. Commun. 14, 2625 (2023).
9. Watson, J. L. et al. Broadly applicable and accurate protein design by integrating structure prediction networks and diffusion generative models. Preprint at doi.org/10.1101/2022.12.09.519842.
10. Hocher, B. et al. Measuring parathyroid hormone (PTH) in patients with oxidative stress—do we need a fourth generation parathyroid hormone assay? PLoS One 7, e40242 (2012).
11. Shackman, J. G., Reid, K. R., Dugan, C. E. & Kennedy, R T. Dynamic monitoring of glucagon secretion from living cells on a microfluidic chip. Anal Bioanal. Chem. 402, 2797-2803 (2012).
12. Baker, M. Reproducibility crisis: Blame it on the antibodies. Nature 521, 274-276 (2015).
13. Bradbury, A. & Plückthun, A. Reproducibility: Standardize antibodies used in research. Nature 518, 27-29 (2015).
14. Bailly, M. et al. Predicting Antibody Developability Profiles Through Early Stage Discovery Screening. MAbs 12, 1743053 (2020).
15. Saper, C. B. A guide to the perplexed on the specificity of antibodies. J. Histochem. Cytochem. 57, 1-5 (2009).
16. Le Basle, Y., Chennell, P., Tokhadze, N., Astier, A. & Sautou, V. Physicochemical Stability of Monoclonal Antibodies: A Review. J. Pharm. Sci. 109, 169-190 (2020).
17. Lee, J. H., Yin, R., Ofek, G. & Pierce, B. G. Structural Features of Antibody-Peptide Recognition. Front. Immunol. 13, 910367 (2022).
18. Cao, L. et al. Design of protein-binding proteins from the target structure alone. Nature 605, 551-560 (2022).
19. Chevalier, A. et al. Massively parallel de novo protein design for targeted therapeutics. Nature 550, 74-79 (2017).
20. Ghirlanda, G., Lear, J. D., Lombardi, A. & DeGrado. W. F. From synthetic coiled coils to functional proteins: automated design of a receptor for the calmodulin-binding domain of calcineurin. J. Mol. Biol. 281, 379-391 (1998).
21. Wright, P. E. & Dyson, H. J. Linking folding and binding. Curr. Opin. Struct. Biot 19,31-38 (2009).
22. Lazar, T., Tantos, A., Tompa, P. & Schad, E. Intrinsic protein disorder uncouples affinity from binding specificity. Protein Si. 31, e4455 (2022).
   23. Gisdon, F. J. et al. Modular peptide binders—development of a predictive technology as alternative for reagent antibodies. Biol. Chem. 403, 535-543 (2022).
24. Wu, K. et al. De novo design of modular peptide-binding proteins by superhelical matching. Nature 616, 581-589 (2023).
25. Grigoryan, G. & Degrado, W. F. Probing designability via a generalized model of helical bundle geometry. J. Mol. Biol. 405, 1079-1100 (2011).
26. Wang, J. et al. Scaffolding protein functional sites using deep learning. Science 377, 387-394 (2022).
27. Dauparas, J. et al. Robust deep learning-based protein sequence design using ProteinMPNN. Science 378, 49-56 (2022).
28. Kobayashi, K. et al. Endogenous ligand recognition and structural transition of a human PTH receptor. Mol. Cell 82, 3468-3483.e5 (2022).
29. Yin, H. et al Computational design of peptides that target transmembrane helices. Science 315, 1817-1822 (2007).
30. Praetorius, F. et al. Design of stimulus-responsive two-state hinge proteins. bioRxiv 2023.01.27.525968 (2023) doi:10.1101/2023.01.27.525968.
31. Jumper, J. et al. Highly accurate protein structure prediction with AlphaFold. Nature 596, 583-589 (2021).
32. Hall, C. M., Glaser, S. & Alpini, G. Gastrointestinal Hormone (GI) Regulated Signal Transduction☆, in Reference Module in Neuroscience and Biobehavioral Psychology (Elsevier, 2017).
33. Anishchenko, I. et al. De novo protein design by deep network hallucination. Nature 600, 547-552 (2021).
34. Wicky, B. I. M. et al. Hallucinating symmetric protein assemblies. Science 378, 56-61(2022).
35. Liu, Q. et al. Apoptotic regulation by MCL-1 through heterodimerization. J. Biol. Chem. 285, 19615-19624 (2010).
36. Crabtree. M. D., Mendonga, C. A. T. F., Bubb, Q. R. & Clarke, J. Folding and binding pathways of BH3-only proteins are encoded within their intrinsically disordered sequence, not templated by partner proteins. J. Biol. Chem. 293, 9718-9723 (2018).
37. Khatib, F. et al. Algorithm discovery by protein folding game players. Proc. Natl. Acad. Sci. U.S.A 108, 18949-18953 (2011).
38. Larhammar, D. Evolution of neuropeptide Y, peptide YY and pancreatic polypeptide. Regul. Pept. 62, 1-11 (1996).
39. Kang, H, er al. Structural basis for Y2 receptor-mediated neuropeptide Y and peptide YY signaling. Structure 31, 44-57.e6 (2023).
40. Sill A. et al. Advancing the immunoaffinity platform AFFIRM to targeted measurements of proteins in serum in the pg/ml range. PLoS One 13, e0189116 (2018).
41. Makaraviciute, A. & Ramanaviciene, A. Site-directed antibody immobilization techniques for immunosensors. Biosens. Bioelectron. 50, 460-471 (2013).
42. Quijano-Rubio, A. et al. De novo design of modular and tunable allosteric biosensors. bioRxiv (2020) doi:10.1101/2020.07.18.206946.
43. Shi, J. et al. A distributable LC-MS/MS method for the measurement of serum thyroglobulin. J Mass Spectrom. Adv. Cin. Lab 26, 28-33 (2022).
44. Hoofnagle, A. N., Becker, J. O., Wener, M. H. & Heinecke, J. W. Quantification of thyroglobulin, a low-abundance serum protein, by immunoaffinity peptide enrichment and tandem mass spectrometry. Clin. Chem. 54, 1796-1804 (2008).
45. Hossain, M. Selected Reaction Monitoring Mass Spectrometry (SRM-MS) in Proteomics: A Comprehensive View. (Springer Nature, 2020).
46. Zhou, H. et al. Generation of monoclonal antibodies against highly conserved antigens. PLoS One 4, e6087 (2009).
47. Rajan, S., Choi, M., Back. K. & Yoon, H. S. Bh3 induced conformational changes in Bcl-XI revealed by crystal structure and comparative analysis. Proteins 83, 1262-1272 (2015).
48. Lee, E. F. et al. High-resolution structural characterization of a helical alpha/beta-peptide foldamer bound to the anti-apoptotic protein Bcl-xL. Angew. Chem. Int. Ed Engl. 48, 4318-4322(2009).
49. Parthier, C. et al. Crystal structure of the incretin-bound extracellular domain of a G protein-coupled receptor. Proc. Natl. Acad. Sci. U S. A. 104, 13942-13947 (2007).
50. Dong, M. et al. Structure and dynamics of the active Gs-coupled human secretin receptor. Nat. Commun. 11, 4137 (2020).
51. Zhang, X. et al. Differential GLP-1R Binding and Activation by Peptide and Non-peptide Agonists. Mol. Cell 80, 485-500.e7 (2020).
52. Jin, L. et al. Crystal structure of human parathyroid hormone 1-34 at 0.9-A resolution. J. Biol. Chem. 275, 27238-27244 (2000).
53. Nygaard, R., Nielbo, S., Schwartz. T. W. & Poulsen, F. M. The PP-fold solution structure of human polypeptide YY and human PYY3-36 as determined by NMR. Biochemistry 45, 8350-8357 (2006).
54. Tang, T. et al Receptor-specific recognition of NPY peptides revealed by structures of NPY receptors. Sci Adv 8, eabm1232 (2022).
55. Wojdyla, J. A., Fleishman. S. J., Baker. D. & Kleanthous, C. Structure of the ultra-high-affinity colicin E2 DNase—Im2 complex. J. Mol Biol. 417, 79-94 (2012).
56. Kühlmann, U. C., Pommer, A. J., Moore, G. R., James, R. & Kleanthous, C. Specificity in protein-protein interactions: the structural basis for dual recognition in endonuclease colicin-immunity protein complexes. J. Mol. Biol. 301, 1163-1178 (2000).
57. Shushan, A. & Kosloff, M. Structural design principles for specific ultra-high affinity interactions between colicins/pyocins and immunity proteins. Sci. Rep. 11, 3789 (2021).

### Computational Methods

### Parametric Design of Groove-Shaped Scaffold Library and Use for Binder Design

The parametric groove-shaped scaffold library was sampled using a random sampling approach, where key parameters' were selected randomly from distributions. An even distribution of bundle “lengths” was sampled, where each parametric helix was 15-19 residues long. A supercoiling value was randomly selected from a biased distribution favoring more supercoiled scaffolds, given these scaffolds were more likely to fail in the subsequent looping step (FIG. 1b). This biased sampling strategy was chosen to achieve a more uniform distribution of supercoiling within the final scaffold library, with sufficient numbers of highly-supercoiled bundles. An average helix neighbor distance value was randomly selected from a normal distribution informed by native protein helical bundle geometries (FIG. 1b). The distance of each helix from its neighbors was independently randomly selected from a much tighter normal distribution centered at the preselected average helix neighbor distance value, to provide some noise within a given scaffold to helix distances and allow for heterogeneous amino acid selections. Values for helix phase and Z displacement were randomly sampled for each helix. The “groove” consisting of 3 helices was first sampled as a helical bundle using the Crick parameterization of alpha-helical coiled coils, around an imaginary central helix where the target was to later be docked. Next, the two buttressing helices were sampled with the same parameterization, but moved radially outward with randomly sampled helix neighbor distances as well as an additional randomly sampled tilt. This process was used to sample a set of 200 k arrangements of 5 helices. Next, the Rosetta™ ConnectChainsMover² was used to loop this set into approximately 135 k successful scaffold backbones. These backbones were designed and filtered using Rosetta™ (including flexible backbone design) to yield a final library of 18 thousand scaffolds. Backbones were filtered on metrics including buried nonpolar surface area per residue, Rosetta™ score per residue, percent alanine, exposed hydrophobics per residue, and Rosetta™ ‘holes’⁴. This library was used to design binders to different helical peptide targets using an adapted version of the miniprotein binder design computational pipeline used by Cao et al⁵, in which only the binder interface was designed and the target was restricted to only rotamer repacking.

Inpainting To sample around an initial putative binder, and to extend binder to make additional contacts with the bound peptide, the RFⱼₒᵢₙₜ Protein Inpainting network was used⁶, in conjunction with ProteinMPNN⁷. Rosetta™ designed binders to PTH, GCG and NPY were used as input to RFⱼₒᵢₙₜ. To generate diversity, additional length was added (randomly and independently sampled) at the loop junctions between the binder helices. Additionally, one whole helix was completely rebuilt by RFⱼₒᵢₙₜ, to further permit diversification. Designs out of RFⱼₒᵢₙₜ were subsequently sequence-redesigned with ProteinMPNN, validated/filtered in silico by AlphaFold™2 (AF2) with initial guess^{8,9}, and subsequently tested experimentally.

### Sequence Threading to Generate Peptide Binders

We started from a library of several thousand all-helical scaffolds bound to designed single helices. We then threaded sequences of peptides of interest onto the bound single helix and filtered to obtain threaded conformations that maximized the number of target sequence positions that formed hydrophobic interactions at the interface to the binder scaffold^{10,11}. The resulting binders were then redesigned in the presence of the threaded target sequence with ProteinMPNN⁷ (forbidding cysteine) and the complex was predicted with AF2 with initial guess^{9,8}. Another round of ProteinMPNN and AF2+initial guess was performed on the AF2 models that passed gate filters. Both rounds had gate filters of interface predicted alignment error (pAE)<10, mean pLDDT>92, pTM score >0.8 and RMSD to input backbone <1.75. AF2 models from both rounds that passed gate filters were further filtered on AF2 metrics and filtered on Rosetta™ metrics to select sequences to order. Sequences were filtered against membrane insertion potential¹², contact_molecular_surface, ddg⁵, interface pAE, and monomer pAE⁹.

### AF2 Hallucination for Flexible Peptide Binder Design

Code for running hallucination with AlphaFold2 were modified from Wicky et al¹³, with custom losses developed to promote binding of the hallucinated protein to the input peptide sequence. AlphaFold2 model_4_ptm was used for all experiments.

Initial sequence sampling: In line with Wicky et al., the initial binder sequence was sampled randomly, with amino acids probabilities corresponding to background amino acid frequencies in BLOSUM62 [cite]. The target sequence (but no template structure) is also provided, separated by a chain break (+32 residue positional index offset). Residues were then mutated, with probabilities related to their background frequency in BLOSUM62. The mutation rate at each step is decayed throughout the trajectory (1250×3 steps, 2500×2 steps, 1250×1 step). More mutations initially helps speed up hallucination, while a lower rate later on allows more gradual refinement. To further speed up convergence, mutations were selectively made to residues with the lowest 50% of AF2 pLDDTs.

### Losses Used for Hallucination:

pLDDT of the bound state: Average pLDDT of the binder-peptide complex
pTM of the bound state: The pTM score of the binder-peptide complex
Radius of gyration: The radius of gyration was calculated as the mean squared distance of residues from the center of mass of the protein. To approximately standardize the scaling with length of the protein, this was empirically normalized by dividing the radius of gyration by the radius of a sphere of volume the length of the hallucinated protein.
Contact probability: Calculated as total probability that a residue in the target is in contact (<8 Å) of the target peptide (the summed probability over the sub-8 Å bins of the distogram output from AF2). This was averaged across all binder residues.
Interface pAE: The mean predicted aligment error (pAE) between the binder and peptide chains.

For all examples shown in this work, the losses were weighted with relative weights of 1:1:0.1:3:5.

Simulated Annealing: To optimize the designed binder, simulated annealing was performed, with a starting temperature of 0.01, and the half-life of the exponential decay set to 500 steps. Mutations were accepted or rejected using the Metropolis criterion. A total of 5000 steps were performed during design.

ProteinMPNN: Previous work has demonstrated that AF2 hallucination yields adversarial sequences that do not work experimentally¹³. However, designs can be rescued with ProteinMPNN redesign of the sequences, 64 sequences were designed per backbone, and were subsequently filtered based on AF2 pLDDT, pTM, RMSD to the design model, RMSD of the monomer to the binder model (without the peptide), and Rosetta™ ddg. The precise values used for filtering were chosen to reduce the set down to 46 designs.

### Partial Diffusion to Optimize Binders

RFDiffusion™ was modified to allow the input structure to be noised only up to a user-specified timestep instead of completing the full noising schedule. The starting point of the denoising trajectory is therefore not a random distribution. Rather, it contains information about the input distribution resulting in denoised structures that are structurally similar to the input (FIG. 2a). The AF2 models of the highest-affinity designs from inpainting for GCG and NPY were used as inputs to partial diffusion. The models were subjected to 40 noising timesteps out of a total of 200 timesteps in the noising schedule, and subsequently denoised. An auxiliary potential minimizing the radius of gyration of the binder-peptide complex was used (described below). A couple of thousand partially diffused designs were generated for each target. The resulting library of backbones were sequence designed using ProteinMPNN and FastRelax, followed by AF2+initial guess⁹. The resulting libraries were filtered on AF2 pAE, pLDDT, RMSD to the design model. RMSD of the monomer to the binder model (without the peptide), and Rosetta™ ddg. The precise values used for filtering were chosen to reduce the set down to 96 designs for each target.

### De Novo Peptide Binder Design Using RF Diffusion

The AF2 model of the PTH peptide in the highest-affinity binder from inpainting was used as input to RFdiffusion™. For Bim, there was no previously designed binder and therefore the crystal structure of Bim¹⁴ (PDB: 6X8O) was used as input. The models were subjected to 200 noising timesteps, and were subsequently denoised. An auxiliary potential minimizing the radius of gyration of the binder-peptide complex was used (described below). A couple of thousand diffused designs were generated for each target. The resulting library of backbones were sequence designed using ProteinMPNN and FastRelax, followed by AF2+initial guess⁹. The resulting libraries were filtered on AF2 PAE, pLDDT. RMSD to the design model, RMSD of the monomer to the binder model (without the peptide), and Rosetta™ ddg. The precise values used for filtering were chosen to reduce the set down to 96 designs for each target.

### Radius of Gyration Potential

RFDiffusion™ enables the use of external guiding potentials during inference which helps design proteins with a certain desired property. The utility of these guiding potentials in designing symmetric oligomers and enzymes, as well as a description of how they are incorporated into the sampling procedure is described in Watson et al. In this work, we take advantage of guiding potentials to minimize the radius of gyration (ROG) of the binder-peptide complex. The ROG is calculated as the root mean square of the distance of all the CA atoms from the centroid. It is more important to apply the potential at the initial denoising steps, and less so towards the end when the quaternary structure is largely fixed. Therefore, the scaling factor with which the gradients are multiplied has a cubic decay over the course of the denoising trajectory.

Training RFDiffusion™ for designing binders to targets from sequence alone

A modified version of RFDiffusion™ was trained to permit the design of protein binders to targets, where only the sequence of the target was specified. The training strategy largely followed the training strategy used for the original RFDiffusion™ model, with some modifications. A summary is provided below.

Overview of “base” RFDiffusion™ Training: RFDiffusion™ is a denoising diffusion probabilistic model (DDPM) fine-tuned from a pre-trained structure prediction model; RoseTTAFold™. RFDiffusion™ is trained with a forward noising process that iteratively, over 200 timesteps, noises residue translations and orientations to distributions that are indistinguishable from random distributions (3D Gaussian distribution and a uniform distribution on SO(3), respectively). RFDiffusio™ n is then trained to reverse this corruption process, predicting the ground truth (₀) at each timestep of prediction. Mean squared error (MSE) losses are used to minimize the error between the forward and reverse processes. Full training details are extensively described in Watson et al.

Modifications to RFDjifuson™ for binder design to sequence alone: RFDiffusion™ was trained on both monomers (<384 amino acids) and heterocomplexes (one chain, denoted the “binder chain”<250 amino acids) from the Protein Data Bank (PDB). Coordinates were scaled by a factor of four, in line with the original RFDiffusion™ model. In 20% of cases, no sequence or structure was provided to the model (for unconditional generation). In the other 80% of cases, 20-100% of the protein was noised. In contrast to RFDiffusion™, however, the structure of up to 50% of the protein (monomer or “non-binder chain”) was noised (diffused), while the sequence of those residues was provided. Thus, RFDiffusion™ learns to condition its predictions on the sequence of part of a protein (the monomer) or of a target to bind to. This version of RFDiffusion™ was trained for seven epochs.

### Computational Filtering

Precise metrics cutoffs changed for each design campaign to get to an orderable set, but largely focused on pAE (<10), plddt (>80) and Rosetta™ ddG (<−40)⁹.

### Experimental Methods

### Gene Construction of Peptide Hormone Binders

The designed protein sequences were optimized to be both expressed in S.cerevisiae and E. coli. Linear DNA fragments (eBlocks, Integrated DNA Technologies) encoding design sequences included overhangs suitable for cloning into pETcon3 vector for yeast display⁵ and Golden Gate cloning into LM627 vector for protein expression¹³. For initial testing hallucinated binders to Bid, binders were cloned into a modified LM627 vector. Specifically, Golden Gate cloning was used to generate sfGFP-Bid-STOP-[Binder]-SNAC-HISx6 assemblies.

### Yeast Display Screening

For the yeast transformation, 50-60 ng of digested pETcon3 with the NdeI and XhoI restriction enzymes and 100 ng of insert (eBlocks™, Integrated DNA Technologies) were transformed into S. cerevisiae EBY100 strain using the protocol described in ref⁵. EBY100 cultures were grown in C-Trp-Ura medium supplemented with 2% (w/v) glucose (CTUG). For induction of expression, yeast cells initially grown in CTUG were transferred to SGCAA medium supplemented with 0.2% (w/v) glucose and induced at 30° C. for 16-24 h. Cells were washed with PBSF (PBS with 1% (w/v) BSA) and labeled for 40 minutes with biotinylated peptide targets at room temperature using without-avidity labeling conditions⁵. After incubation time, cells were washed and resuspended in PBSF for individual sorting of cells harboring each unique design using a 96-well compatible autosampler in the Attune™ N×T Flow Cytometer (Thermo Fisher Scientific).

### NanoBiT Screening

Linear gene fragments encoding binder design sequences and target peptide sequences were cloned into E. coli expression vectors using Golden Gate assembly; these vectors were pET28b(+) derivatives genetically fusing the smBiT and lgBiT halves of the NanoLuc® Luciferase (Promega) to the binders and peptides respectively. Resulting plasmids were transformed into BL21* (DE3) (Invitrogen) E. coli competent cells, then grown in 1 mL TBII in 96-deepwell plates at 37 C and 600 rpm. After 2 hours, expression was induced with IPTG (0.1 mM) and cells were incubated for an additional 4 hours. Cells were harvested by centrifugation (15 min at 4 kg), then resuspended in 100 uL lysis buffer (10 mM NaP pH 7.4, 150 mM NaCl, 5 mM MgCl₂, 1 mg/mL lysozyme, 10 ug/mL DNAse 1, 1 tablet Complete Protease inhibitor/50 mL). Cells were incubated for 1 hour at room temperature and 600 rpm, then frozen (−80 C for 30 min) and thawed (37 C at 600 rpm for 30 min) twice. Lysate was cleared by centrifugation (20 min at 4 kg), and the soluble fraction was then transferred to a 96-well plate for use as stock protein/peptide for conducting the nanoBiT screen. Screens were assembled in 96-well Half Area Black Flat Bottom Polystyrene NBS Microplates (Corning 3686). Binder design smBiT lysate was diluted 12 uL into 1400 uL assay buffer (10 mM NaP pH 7.4, 150 mM NaCl), while target peptide lgBiT lsate was diluted 6 uL into 1400 uL assay buffer. Stock rows in the assay plate were prepared by mixing 40 uL substrate (499.2 uL assay buffer, 20.8 uL Nano-Glok Luciferase Assay Substrate (Promega)) with 40 uL diluted binder design smBiT lsate, while experimental rows were prepared by adding 50 uL diluted target peptide lgBiT lysate. At read time, 50 uL of the stock row was added to the 50 uL experimental row and mixed quickly and carefully, then luminescence was read immediately for 5 min using a plate reader (Biotek Synergy Neo2).

Identification of Weak Binder Hits from Parametric Designs

The first helical peptide binder hits were identified in experiments screening for binding using the nanoBiT split luciferase assay. These kinetic binding experiments were performed in cell lysate with no control over protein concentration, so candidate binders were selected qualitatively for showing some increase in luminescence signal over time above background noise, indicating likely binding activity. Additional binding curve experiments indicated that this binding activity was at very weak affinities, likely >100 nM (indistinguishable from the background signal of weak luciferase binding for the assay). Therefore, these initial candidates were not further characterized, but rather selected for additional design to yield higher affinity binders.

### Bicistronic Protein Expression

Hallucinated binders to Bid were screened by bicistronic expression with the Bid peptide. Plasmids encoding sfGFP-Bid-STOP-[Binder]-SNAC-HISx6 were cloned into E. coli, and 2 mL cultures of each of the 47 designs were grown overnight in LB. Cultures were diluted into TB medium, and grown to approximately OD280 0.6, before induction with 1 mM IPTG for 4 hours at 37° C. Bacteria were lysed for 15 minutes in 300 B-PER (Thermo)+1 mM PMSF, 0.1 mg/mL Lysozyme (Sigma), 0.01 mg/mL DNAse I. Lysates were clarified by centrifugation at 4000 g for 10 minutes, before purification on Ni-NTA resin (wash buffer: 20 mM Tris pH 8.0, 150 mM NaCl, 20 mM Imidazole; elution buffer: 20 mM Tris pH 8.0, 150 mM NaCl, 250 mM Imidazole). Eluates were assessed for GFP fluorescence on a fluorescence plate reader.

### Peptide Synthesis and Purification

The PTH-TAMRA peptide was synthesized in-house on a CEM Liberty™ Blue microwave synthesizer. All L- and D-amino acids were purchased from P3 Biosystems. Oxyma Pure™ was purchased from CEM, DIC was purchased from Oakwood Chemical, diisopropyl ethylamine (DIEA) and piperidine were purchased from Sigma-Aldrich. Dimethylfonmamide (DMF) was purchased from Fisher Scientific and treated with an Aldraamine trapping pack prior to use. Synthesis was done on a 0.1 mmol scale on CEM Cl-TCP(CI) resin. Five equivalents of each amino acid were activated using 0.1 M Oxyma with 2% (v/v) DIEA in DMF, 15.4% (v/v) DIC, and coupled on resin for 4 min with double coupling if needed. This was followed by deprotection using 5 mL of 20% piperidine in DMF for 2 min at 95° C. Global deprotection was accomplished TFA/Water/TIPS (95:2.5:2.5) for 3 hours. This deprotection mixture was precipitated in 30 mL of ice-cold ethyl ether, centrifuged and decanted, then washed twice more with fresh ether and dried under nitrogen to yield crude peptide for high-pressure liquid chromatography (HPLC) purification.

The TAMRA-Bid peptide was synthesized using a PurePep™ Chorus peptide synthesizer (Gyros Protein Technologies). Fmoc-protected amino acids were purchased from Ambeed. The peptide synthesis was performed at a 0.05 mmol scale on Rink amide AM resin (Matrix Innovation). Following reagents were prepared in dimethylformamide (DMF, VWR) for amino acid coupling: Fmoc-protected amino acids (0.2 M), O-(1H-6-Chlorobenzotriazole-1-yl)-1,1,3,3-tetramethyluronium hexafluorophosphate (HCTU, 0.5 M. Ambeed), and diisopropyl ethylamine (DIEA, 1.0 M, Thermo Scientific). For peptide elongation, the resin was washed with DMF (3×3 mL) before fmoc-deprotection was carried out using 3 mL 20% (v/v) piperidine (Sigma-Aldrich) in DMF (2×, 2 min, room temperature, agitating at 300 rpm). After a DMF washing step (3×3 mL), amino acid/HCTU/DIEA (1:1.25:2.5) was added to the resin in 5-fold excess and agitated (2×10 min, 50° C.). After a final fmoc-deprotection, N-terminal coupling of 5-(and-6)-carboxytetramethylrhodamine (TAMRA, Anaspec Inc.) was accomplished using following conditions: TAMRA:benzotriazol-1-yloxy)tripyrrolidinophosphonium hexafluorophosphate (PyBOP, Novabiochem):DIEA (1.5:1.5:3) in N-Methyl-2-pyrrolidone (NMP. Sigma-Aldrich) for 16 h at room temperature and 300 rpm. Tin foil was used to cover the reaction vessel to avoid photobleaching of the fluorophore. For global deprotection and cleavage from the resin, the peptide was incubated with TFA/Water/triisopropyl silane (TIPS, Sigma-Aldrich)/1,2-Bis(2-mercaptoethoxy)ethane, 3,6-Dioxa-1,8-octane-dithiol (DODT, Sigma-Aldrich) (92.5:2.5:2.5:2.5) for 2 h at room temperature while agitating, 30 mL of ice-cold diethyl ether (VWR) was added for peptide precipitation and the precipitated peptide was centrifuged at 2500×g for 10 min at 4° C. The supernatant was discarded, and the peptide was washed with 20 mL of diethyl ether. The peptide pellet was dissolved in water/acetonitrile/TFA (50:50:0.1) and lyophilized.

The crude peptide was dried and dissolved in a mixture of ACN and water where the entire crude is soluble. This solution was purified on a C18 column in an Agilent HPLC instrument. A linear gradient of increasing ACN with 0.1% TFA was used to purify the samples. UV signal was monitored at 214 nm and all peaks were collected. Peaks were checked using ESI mass spectroscopy for the correct peptide mass. The purified peptide was then lyophilized for further use.

Protein Expression and Purification in E. coli for Peptide Hormone Binders

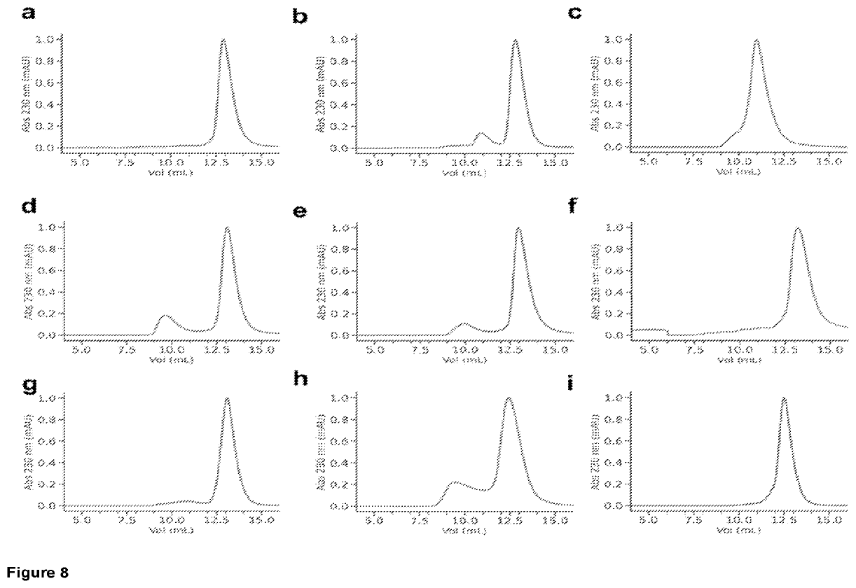

Protein expression was performed using 50 mL of the Studier autoinduction media supplemented with kanamycin, and grown overnight at 37° C. The cells were harvested by spinning at 4,000×g for 10 min and then resuspended in lysis buffer (100 mM Tris-HCl, 200 mM NaCl, 50 mM imidazole) supplemented with protease inhibitor tablets (Pierce™ Protease Inhibitor Tablets. EDTA-free). Then, the cells were lysed by sonication in a Qsonica, Q500 with a: 4-pronged horn for 2:30 min ON total, with an amplitude of 80%. Soluble fractions were clarified by centrifugation at 14,000×g for 40 minutes, and were subsequently purified by affinity chromatography using bed Ni-NTA resin (Qiagen or Thermo Fisher) on a vacuum manifold. A series of washes using Low-salt buffer (20 mM Tris-HCl, 200 mM NaCl, 50 mM imidazole) and High-salt buffer (20 mM Tris-HCl, 1000 mM NaCl, 50 mM imidazole) were performed prior to elution with Elution buffer (20 mM Tris-HCl, 200 mM NaCl, 500 mM imidazole). After elution, protein samples were filtered and injected into an autosampler-equipped Akta pure system on a Superdex™ S75 Increase 10/300 GL column at room temperature. The SEC running buffer was 20 mM Tris-HCl, 100 mM NaCl pH 8. We pooled the largest abundance monodisperse peak fractions and concentrated using Spin filters (3 kDa molecular weight cutoff, Amicon, Millipore Sigma) and stored at 4° C. before downstream characterizations. Protein concentrations were determined by absorbance at 280 nm using a NanoDrop™ spectrophotometer (Thermo Scientific) using their extinction coefficients and molecular weights obtained from their amino acid sequences using the ProtParam™ tool. We additionally verified the monodispersity of the pooled fractions by reinjecting them on the same column for the majority of the binders (FIG. 8)

### Fluorescence Polarization

Fluorescence polarization binding assays were carried out in 96-well plates (Corning 3686), with two-fold serial dilution of designed peptide binders in the presence of 0.5 nM fluorescently labeled peptide targets. Protein and peptide were diluted from their stock concentration into 20 mM Tris-HCl pH 8, 100 mM NaCl, 0.1% v/v Tween 20, and the protein was titrated in 2-fold serial dilutions onto constant peptide. After incubating the peptide and binder for one hour at room temperature, the fluorescence polarization was measured at the excitation and emission wavelengths of the FAM dye (485/530 nm) or the TAMRA dye (530/590 nm), in a Synergy Neo2 multi-mode plate reader. Titrations were conducted in replicate, and the Kd was fitted with SciPy¹⁸. Specifically, curves were fit to N observations of an observed signal, Signalᵢ, at titrated concentrations according to the following equation:

$${Signal}_{i} = Baseline + {AmplitudeAB}_{conc} ,$$

Where [Bₜₒₜ] is the known total concentration of the binder, Baseline and Amplitude are free parameters, and the concentration of the bound state [AB] is computed as

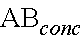

The unknown parameters (K_{D}, Baseline and Amplitude) were fit using scipy.optimize.curve_fit, [Bₜₒₜ] was additionally fit in the optimization, but only allowed to within 0.5 nM±0.1%.

Fluorescence polarization measurements for TAMRA-Bid were performed in 96-well, flat-bottom, half-area microplates (Corning 3881) using a CLARIOstar Plus plate reader (BMG Labtech) set to excitation and emission wavelengths of 540 and 590 nm, respectively. TAMRA-Bid peptide and hallucinated proteins were diluted into a buffer containing 50 mM sodium phosphate pH 7.0 and 0.05% v/v Tween-20. The concentration of TAMRA-Bid peptide was kept constant at 10 nM. For the proteins, a 2-fold serial dilution was performed and added to the peptide. After one to four hours of incubation at room temperature, FP measurements were conducted. Titrations were carried out in Triplicates.™

### Cloning, Expression and Purification of Bid-Binding Hallucinations, Avi-Tagged Bid Peptide and MCL-1

Bid-binding hallucinations were cloned into a pET28 vector, containing an N-terminal His₁₀ and a PreScission™ cleavage site, using TEDA cloning¹⁹ and transformed into XL-l-Blue chemically competent cells, single clones isolated and amplified and sequences confirmed by Sanger sequencing. Plasmids transformed into chemically competent BL21 DE3 E. coli, and plated onto LB agar plates supplemented with 100 μg/mL kanamycin. Single colonies were used to make starter cultures of LB with 100 μg/mL kanamycin and incubated ovemight at 37° C. 1:100 volume starter culture was added to autoinduction media Overnight Express Instant TB Medium (Novagen) in Ultra-Yield™ flasks (Thomson), with 100 μg/mL kanamycin, incubated at 37° C. for 5 hours, then 18° C. for 18 hrs. Cells harvested by centrifugation 6,000 rpm, 20 mins, 4° C., and pellets were frozen at −80° C.

Defrosted cell pellets were resuspended in approx. 10 mL/g Lysis Buffer (50 mM potassium phosphate pH 7.0, 300 mM NaCl, 5 mM imidazole, 2 mM b-mercaptoethanol, 10% glycerol), supplemented with 60 μg/mL lysozyme, 1.4 pg/mL DNaseI, 0.05 mM PMSF. Cells were lysed by passing through French press twice, 18 kpsi. Lysate was clarified by centrifugation 18,000 g, 45 mins, 4° C. and loaded onto HIS-Select Nickel affinity resin (Sigma) by gravity, resin washed with Wasg Buffer (50 mM potassium phosphate pH 7.0, 100 mM NaCl, 5 mM imidazole, 2 mM b-mercaptoethanol, 10% glycerol) and eluted with Wash Buffer containing 350 mM imidazole. Protein containing fractions (assessed by AMo) were combined, and further purified by size exclusion chromatography (SEC) using HiLoad™ 16/600 200 pg Superdex™ column (Cytiva) using ÅKTA FPLC system (Cytiva) equilibrated in 50 mM sodium phosphate pH 7.0, 1 mM DTT. Fractions were concentrated, concentration measured using A₂₈₀ and predicted extinction coefficients³³, then flash frozen N₂₍ₗ₎ for storage at −80° C.

DNA corresponding to BH3 motif of human Bid Q79-G144 (Uniprot: P55957) was assembled by complementary oligos (IDT) and primer extension using Klenow fragment (NEB), and cloned using TEDA into pET28 with an N-terminal His₁₀, SUMO and C-terminal Avi. Expression and purification was carried out as for the hallucinations, except for co-transformation with a chloramphenicol-resistant BirA expressing plasmid, the addition of chloramphenicol 25 ug/mL in all cultures, with the addition of 40 μM BTN to the media before temperature was reduced to 18° C. After SEC, His₁₀-SUMO was cleaved using ULP-1 protease, and His₁₀-SUMO removed using Ni resin, Bid-Avi peptide concentration was measured using A₂₈₀, and stored at −80° C. To express human Mcl-1 P166-G327 (Uniprot: Q07820) a pEQ80L vector with N-terminal His₆ and Avi-tag, for co-expression with BirA. Expression and purification was carried out as for the Bid-binding hallucinations, with the addition of 40 μM BTN to the media before temperature was reduced to 18° C.

### Isothermal Titration Calorimetry

Isothermal titration calorimetry was carried out with an ITC200 (Micocal). Bid peptide was in the syringe, at ~300 μM, and binder (hallucination of Mcl-1) was kept in the cell (~25 μM), with both peptide and binder in matched buffer (sodium phosphate pH 7.0, 1 mM DT). Temperature was held at 25° C. or 10° C., as indicated. Fitting of titrations was carried out using 1-site binding, using manufacturers software (OriginLab).

Circular Dichroism Spectra were recorded for Bid peptide alone, Bid in complex with binders (hallucination or Mcl-1) and binders alone. All concentrations were 10 μM, in a 2 cm pathlength quartz cuvette. Spectra recorded on J-1500 Circular Dichroism Spectrophotometer, with temperature held at 25° C., or ramped at I ° C./min.

### Pull-Down

10 μL bead slurry Dynabeads™ M-280 Streptavidin (Thermo Fisher Scientific) were washed with Pull-Down Buffer (sodium phosphate pH 7.0, 1 mM DTT, 0.05% Tween20), incubated with saturating amounts of (Avi-tagged) Bid peptide 15 mins, 4° C. with rotation, beads were then incubated with free biotin 25 μM, and washed three times with ice cold Pull-Down Buffer, 10 μL of 2 μM binder (hallucination or Mcl-1) was incubated with pelleted beads for 30 mins, 4° C., with rotation. Supernatant was recovered and the beads washed three times before resuspension in 10 μL Pull-Down Buffer. Both supernatant and washed beads were loaded onto denaturing SDS-PAGE, with protein detection by InstantBlue™ Coomassie staining.

### Crystallization and Structure Determination

All crystallization experiments were conducted using the sitting drop vapor diffusion method. Crystallization trials were set up in 200 nL drops using the 96-well plate format at 20° C. Crystallization plates were set up using a Mosquito LCP from SPT Labtech, then imaged using UVEX microscopes and UVEX PS-256 from JAN Scientific. Diffraction quality crystals formed in 0.2 M Ammonium chloride 0.1 M Tris pH 8 20% (w/v) PEG 6000 for GCG_partdiff; in 0.9 M Halogens, 0.1 M Tris-Bicine pH 8.5 Buffer, and 37.5% of 25% v/v MPD; 25% PEG 1000; 25% w/v PEG 3350 mixture for GCG_inpaint. In 0.1 M Citric acid pH 2.5, 20% (w/v) PEG 6000. PHT 0.2 M Sodium chloride for Bim_fulldiff, and in 0.1 M Sodium acetate pH 4.5, and 1.26 M Ammonium sulfate for PTH peptide only.

Diffraction data was collected at the Advanced Photon Source (APS) beamline 24-ID-C. X-ray intensities and data reduction were evaluated and integrated using XDS²⁰ and merged/scaled using Pointless/Aimless in the CCP4 program suite²¹. Starting phases were obtained by molecular replacement using Phaser²² using the designed model for the structures. Following molecular replacement, the models were improved using phenix.autobuild²³; efforts were made to reduce model bias by setting rebuild-in-place to false, and using simulated annealing and prime-and-switch phasing. Structures were refined in Phenix²³. Model building was performed using COOT²⁴. The final model was evaluated using MolProbity²⁵. Data collection and refinement statistics are recorded in Table 4.

Design and Characterization of lucCagePTH Biosensor for Parathyroid Hormone Detection

The detailed design protocol for the lucCage and lucKey sensor system was described previously²⁶. In brief, the amino acid sequence (FELLDKLIELLRELIELTREYI; SEQ ID NO:13) at the N-terminal end of the 6.1 nM PTH binder was grafted onto the latch region (residues 323 to 353) of lucCage. The Rosetta™ models were visually inspected and eight of them were selected for experimental validation. We produced, purified, and screened for the luminescence signal emitted from each biosensor in the presence of 5 μM PTH. From this process, we identified several hits showing increased luminescence upon adding PTH, of which we assigned the best one with a 21-fold activation as lucCagePTH. We then set up assays to evaluate the response of lucCagePTH with a range of PTH concentrations, 10 μl of 10 nM lucCagePTH, 10 μl of 10 nM lucKey, 10 μl of serial diluted PTH, and 40 μl of buffer (50% HBS-EP/50% Nano-Glo luciferase assay buffer) were pre-mixed and 30 μl of 100× diluted furimazine was injected immediately before luminescence kinetic acquisition. The luminescence measurements were taken every 1 min (0.1 s integration and 10 s shaking during intervals) for a total of 60 mins by Neo2 microplate reader. The linear region of luminescence responses to the corresponding PTH concentrations was fitted to a linear regression curve and the LOD was calculated as 3×standard deviation of the response/the slope of the calibration curve.

### Affinity Enrichment of PTH Analyzed by LC-MS/MS

### Sample Description

Recombinant human PTH protein was purchased from Sigma (#SAE 0192_100 ug, MA, USA) and reconstituted at 100 μg/mL in a 10% acetonitrile, 0.1% formic acid, 1 mg/mL bovine serum albumin solution and stored in 40 μL aliquots at −20° C. Dilutions at 1000 ng/mL and 62.5 ng/mL were prepared freshly as needed by dilution in the same acetonitrile, formic acid, albumin solution.

The plasma samples used were de-identified clinical samples obtained from the clinical laboratories at the University of Washington Medical Center. The use of de-identified leftover clinical samples was reviewed by the University of Washington Human Subjects Division (STUDY0013706).

The evaluation of PTH immunoaffinity enrichment in buffer and plasma was performed in three process replicates using 8 different types of samples:
Series A: Reconstitution buffer (10% acetonitrile, 0.1% formic acid, 1 mg/mL bovine serum albumin in water) served as the blank.
Series B: Reconstitution buffer spiked with PTH at 7.2 ng/mL was directly digested without the addition of beads and served as the Control sample (representing 100% recovery of PTH).
Series C: Reconstitution buffer spiked with PTH at 7.2 ng/mL was incubated with beads blocked by bovine serum albumin before washing and digestion, which served as the negative control, to quantify non-specific binding in buffer.
Series D: Reconstitution buffer spiked with PTH at 7.2 ng/mL was incubated with designed binder-conjugated beads before washing and digestion, which was used to quantify the affinity precipitation of PTH from buffer.
Series E: Plasma was incubated with beads blocked by bovine serum albumin before washing and digestion, which was used to quantify non-specific binding in unspiked plasma.
Series F: Plasma was incubated with designed binder-conjugated beads before washing and digestion, which was used to quantify affinity precipitation of PTH in plasma.
Series G: Plasma spiked with PTH at 7.2 ng/mL was incubated with beads blocked by bovine serum albumin before washing and digestion, which was used to quantify non-specific binding in spiked plasma.
Series H: Plasma spiked with PTH at 7.2 ng/mL was incubated with designed binder-conjugated beads before washing and digestion, which was used to quantify the affinity precipitation of PTH in spiked plasma.

### Sample Preparation and LC-MS/MS Conditions

Affinity enrichment was performed in buffer or plasma at the protein level. Designed binders were conjugated to tosyl-activated Dynabeads™ M-280 according to the manufacturer's instructions and subsequently blocked using bovine serum albumin and Tris. The amino terminal peptide was analyzed after tryptic digestion of either pure protein in buffer, or after trypsin digestion of PTH that had been affinity precipitated by the designed binder-conjugated beads (or by the control/blocked magnetic beads). Briefly, PTH proteins in buffer/plasma were purified using PTH mini-binder conjugated-paramagnetic beads at room temperature, for 1 h. The beads were then washed 4 times with phosphate-buffered saline supplemented with CHAPS (0.1% 3-((3cholomidopropyl) dimethylammonio)-1-propanesulfate to reduce nonspecific interactions). The proteins that were affinity precipitated by the designed binder-conjugated-paramagnetic beads were suspended in 10 μL of a solution containing 10% acetonitrile, 0.1% formic acid, I mg/mL bovine serum albumin. The washed beads were then suspended with 30 μL of 30% isopropanol, 100 mM ammonium bicarbonate, and digested at 37° C. for 30 min after adding 100 μL of 0.01 mg/mL trypsin in 10 mM hydrochloride acid. The liberated peptides were then removed from the beads using a magnet and analyzed using LC-MS/MS.

Peptides were analyzed by liquid chromatography-tandem mass spectrometry in the multiple reaction monitoring acquisition mode using an UHPLC I-Class Chromatography system coupled to a Xevo™ TQ-S triple quadrupole tandem mass spectrometer (Waters, MA, USA). Peptides were eluted from an Acquity™ UPLC HSS T3 1.8 μm (C18, 2.1×50 mm, pore size 100 Å) analytical column (Waters) at 45° C. using 0.1% formic acid, 2% dimethylsulfoxide in LC-MS grade water as mobile phase A and 0.1% formic acid, 2% dimethylsulfoxide in LC-MS grade methanol as mobile phase B.

The liquid chromatography and mass spectrometry conditions are detailed in Tables 5-6.

TABLE 5
Liquid chromatography conditions

| | |
|---|---|
| Mobile phase | Phase A: 0.1% formic acid, 2% dimethylsulfoxide |
| | in water 0.1% formic acid, 2% dimethylsulfoxide |
| | in methanol |
| Column | Acquity ™ UPLC HSS T3 1.8 μm (C18, 2.1 × |
| | 50 mm, pore size 100 Å) |
| Temperature | 45 ± 5° C. |
| Flow rate | 0.3 mL/min |
| Injection volume | 20 μL |
| Gradient | 0-0.5 min: 2% B at 0.3 mL/min |
| | 7.5: 98% B at 0.3 mL/min |
| | 7.6: 98% B at 0.6 mL/min |
| | 8.6: 2% B at 0.6 mL/min |
| | 9.9: 2% at 0.3 mL/min |

TABLE 6
Mass spectrometry conditions

| Source polarity | ESI+ | |
|---|---|---|
| Capillary voltage | 3.25 | kV |
| Source Offset voltage | 50 | V |
| Desolvation Temp | 600° | C. |
| Desolvation Gas Flow | 1000 | L/h |
| Cone Gas Flow | 150 | L/h |

### Data Treatment

Data processing was performed with Skyline Daily version 21.1.1.223. The peak area for each peptide was determined as the sum of the peak are—as of all selected transitions. The recovery over blocked-beads (RE) in spiked buffer and in spiked plasma was estimated using Equations 1, and 2, respectively.

$${RE}_{buffer} = \frac{Peak area Series D}{Peak area Series B} \qquad ( 1 )$$

$${RE}_{plasma} = \frac{Peak area Series H}{Peak area Series B} \qquad ( 2 )$$

### REFERENCES

1. Grigoryan, G. & Degrado, W. F. Probing designability via a generalized model of helical bundle geometry. J. Mol. Biol. 405, 1079-1100 (2011).
2. Brunette, T. J. et at. Exploring the repeat protein universe through computational protein design. Nature 528, 580-584 (2015).
3. Leman, J. K. et al. Macromolecular modeling and design in Rosetta: recent methods and frameworks. Nat. Methods 17, 665-680 (2020).
4. Rocklin, G. J. et 71. Global analysis of protein folding using massively parallel design, synthesis, and testing. Science 357, 168-175 (2017).
5. Cao, L. et al. Design of protein-binding proteins from the target structure alone. Nature 605, 551-560 (2022).
6. Wang, J. et al. Scaffolding protein functional sites using deep learning. Science 377, 387-394 (2022).
7. Dauparas, J. e at Robust deep learning-based protein sequence design using ProteinMPNN. Science 378, 49-56 (2022).
8. Jumper, J. et al. Highly accurate protein structure prediction with AlphaFold. Nature 596, 583-589 (2021).
9. Bennett, N. R. et al. Improving de novo protein binder design with deep learning. Nat. Commun. 14, 2625 (2023).
10. Yin, H. et al. Computational design of peptides that target transmembrane helices. Science 315, 1817-1822 (2007).
11. Ghirlanda. G., Lear, J. D., Lombardi, A. & DeGrado, W. F. From synthetic coiled coils to functional proteins: automated design of a receptor for the calmodulin-binding domain of calcineurin. J. Mol. Biol. 281, 379-391 (1998).
12. Wang, J. Y. J. et al. Improving the secretion of designed protein assemblies through negative design of cryptic transmembrane domains. Proc. Natl. Acad. Si. U.S.A 120, e2214556120 (2023).
13. Wicky, B. I. M. et al. Hallucinating symmetric protein assemblies. Science 378, 56-61 (2022).
14. Assafa, T. E. et al. Biophysical Characterization of Pro-apoptotic BimBH3 Peptides Reveals an Unexpected Capacity for Self-Association. Structure 29, 114-124.e3 (2021).
15. Watson, J. L. et al. Broadly applicable and accurate protein design by integrating structure prediction networks and diffusion generative models. bioRxiv 2022.12.09.519842 (2022) doi:10.1101/2022.12.09.519842.
16. Back, M, er al. Accurate prediction of protein structures and interactions using a three-track neural network. Science 373, 871-876 (2021).
17. Back, M. et al. Efficient and accurate prediction of protein structure using RoseTTAFold2. bioRriv 2023.05.24.542179 (2023) doi: 10.1101/2023.05.24.542179.
18. Virtanen, P. et al. SciPy 1.0: fundamental algorithms for scientific computing in Python. Nat. Methods 17, 261-272 (2020).
19. Xia, Y. et al. T5 exonuclease-dependent assembly offers a low-cost method for efficient cloning and site-directed mutagenesis. Nucleic Acids Res. 47, e15 (2019).
20. Kabsch, W. XDS. Acta Crystallogr D Biol. Crystallogr. 66, 125-132 (2010).
21. Winn, M. D. et al. Overview of the CCP4 suite and current developments. Acta Crystallogr D Biol. Crystallogr 67, 235-242 (2011).
22. McCoy, A. J. et al. Phaser crystallographic software. J. Appl. Crystallogr 40, 658-674 (2007).
23. Adams, P. D. et al. PHENIX: a comprehensive Python-based system for macromolecular structure solution. Acta Crystallogr. D Biol. Crystallogr 66, 213-221 (2010).
24. Emsley, P. & Cowtan, K. Coot: model-building tools for molecular graphics. Acta Crystallogr D Biol. Crystallogr 60, 2126-2132 (2004).
25. Williams, C. J. et al. MolProbity: More and better reference data for improved all-atom structure validation. Protein Sci. 27, 293-315 (2018).
26. Quijano-Rubio, A. et al. Dc novo design of modular and tunable protein biosensors. Nature 591, 482-487 (2021).

1. A polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-12.

2. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1, 2, and 10, wherein the polypeptide binds to parathyroid hormone (PTH).

3. The polypeptide of claim 2, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:2, wherein at least 1, 2, 3, 4, 5, 6, 7, 8, 9, 10, 11, 12, 13, 14, 15, 16, 17, 18, 19, 20 or all of residues 2, 12, 13, 15, 16, 19, 20, 23, 60, 70, 71, 74, 75, 76, 77, 79, 122, 126, 129, 130, and 133 are identical relative to SEQ ID NO:2.

4. The polypeptide of claim 2, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:10, wherein at least 1, 2, 3, 4, 5, or all of residues 7, 8, 15, 18, 53, and 71 are identical relative to SEQ ID NO:10.

5. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:3 and 8, wherein the polypeptide binds to glucagon (GCG).

6. The polypeptide of claim 5, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:3, wherein at least 1, 2, 3, 4, 5, 6, or more, or all of residues 6, 13, 83, 96, 97, 99, 100, 101, 143, 146, 147, 161, 162, and 164 are identical relative to SEQ ID NO:3.

7. The polypeptide of claim 5, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:8, wherein at least 1, 2, 3, 4, 5, 6, 7, 8, 9, 10, 11, 12, 13, 14, 15, or all of residues 16, 17, 20, 24, 28, 79, 82, 83, 86, 143, 144, 146, 147, 150, 157, and 161 are identical relative to SEQ ID NO:8.

8. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:4 and 9, wherein the polypeptide binds to neuropeptide Y (NPY).

9. The polypeptide of claim 8, comprising
(a) an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:4, wherein at least 1, 2, 3, 4, 5, 6, 7, 8, 9, 10, 11, 12, 13, 14, 15, 16, or all of residues 16, 17, 18, 21, 26, 27, 28, 43, 53, 73, 93, 100, 103, 106, 135, 146, and 150 are identical relative to SEQ ID NO:4; or
(b) an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:9, wherein at least 1, 2, 3, 4, 5, 6, 7, or all of residues 24, 73, 133, 145, 150, 151, 156, and 157 are identical relative to SEQ ID NO:9.

10. (canceled)

11. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:5, wherein the polypeptide binds to secretin (SCT).

12. (canceled)

13. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:6, wherein the polypeptide binds to gastric inhibitory peptide (GIP).

14. (canceled)

15. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7, wherein the polypeptide binds to glucagon-like peptide 1 (GLP1).

16. (canceled)

17. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:11, wherein the polypeptide binds to BIM BH3 peptide (Bim).

18. (canceled)

19. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:12, wherein the polypeptide binds to peptide YY (PYY).

20.-22. (canceled)

23. A fusion protein, comprising:
(a) the polypeptide of claim 1; and
(b) one or more functional domains.

24. A nucleic acid encoding the polypeptide of claim 1.

25. An expression vector comprising the nucleic acid of claim 24 operatively linked to a suitable control sequence.

26. A host cell comprising the expression vector of claim 25.

27. A pharmaceutical composition, comprising:
(a) the polypeptide of claim 1; and
(b) a pharmaceutically acceptable carrier.

28. A method, comprising contacting a biological sample with the polypeptide of claim 1, and detecting binding of the polypeptide with target present in the biological sample, wherein presence of the target in the biological sample correlates with a disease state.


SEQUENCE LISTING (16 sequences)

SEQ ID NO: 1 - protein, 113 aa, synthetic construct
SQELIEELIKLAKELAEIKDEEERRKIKRELERLAEELKEAPASSLLRALALLVIALALIQAAESEEERERARELLERLEELLRELEKQITDERFKEILRELEELAKELKKQL

SEQ ID NO: 2 - protein, 136 aa, synthetic construct
SFELLEKLIELSKELYEVAKKYIETGDPELKKKLEEILKKIEEAYKELLESDAHPLEKALAKLILAEAYVVKSFAYISSGKDLEKAQEYLDKAKEILEELLKLLEELKKEETDPEKLEIIEELEKIAEELLKEIEE

SEQ ID NO: 3 - protein, 168 aa, synthetic construct
MLDELFSLLNKMFELSDKYRELRKELRKAIESGAPEEELRELLEKMLEIAKKLLELTKELKKLVEDVLKNNPDPVERAKAVLLYAVGVHILYSESSELEVIAERLGFKDIAEKAKEIADKARELKEEVKRKLREIREEVPDPEIRKAAEEAIEMLESNDKRLKEFRKL

SEQ ID NO: 4 - protein, 161 aa, synthetic construct
GAAEKLAELYEKFKALREKALEVLKKAVEALENKADKETLLKLIKELKELAEKFEELAEEFERNAGESTTASLNATAAYMARIGLLAALLALAKAAGVPEEELEEIKRRIEETAKRAIEAAERLKALAEARGDTKHVAVGVEAVRMATELYELAQKIIDAF

SEQ ID NO: 5 - protein, 221 aa, synthetic construct
SEELEERLREARERLEEARERLEEAREEGDLREMARALLEEARAVLEIARVAAEAGDDEALREAARRAGEVIRRAGEVGLRAAEEGDTETIREAMLAILEAQRASAVIALHLARDDPEVAEALRVIERLLRTAERALREGQLEVARLATEAVEALADAILRAREIGRPELVREAARLAEEARRLLEAALEALRAGDEEGARERLARARELIREIRERVRRA

SEQ ID NO: 6 - protein, 224 aa, synthetic construct
SPKEKAERLIKEAKEAAEKAKEAAERSGLEEAKKAAEELTKLLEEAAARVAADPEDETKLRALEKIVEAAKEAVKALEVAIESGDEQLIRAALSLVEAAVHLAKALLAKPESPLVDFGFELLKLAAKTLAAYAEGEDVDKIALKLKAISAMAEALRLALAGDLERAARAAEEAVRYAIEAGDKELLRLAAEVAAYIARLAEEAGLEEVARRAREAAERAREAAK

SEQ ID NO: 7 - protein, 203 aa, synthetic construct
SPEEEARRAAREAERAAREAREAARRLGDEESVRVAERLEREARRAERERDLELARRVLRAAEALRLALEGELLAREQGDELGVVVARMITLAARDSALGRGTPELARLLLRVARALLEGDLEEVVRSLAEIAKREIGTERALLAVEAIKLVALESIEEGDFETAELAIEKLREIAEEFEGTEVAEKAREAIEEIEKKKREAE

SEQ ID NO: 8 - protein, 168 aa, synthetic construct
SMEKLAEIMQEIIEAYQEVKDAFFKFIKAVHEGAPEEELKKYLEKMKEALEKMKELLERLEKEAKKVIEENKDKKLELKVLLMLRLAYLLLKVSIELTKIAAEKLGDKELVEELEKESKEVEKKIKELEERIKKLLEEVDDEELKEAYKEVEEMEKEAEKFLEKMRKV

SEQ ID NO: 9 - protein, 161 aa, synthetic construct
GMEERRKELLEKLKKLKEEVVELFRELAQALRDGASKERLEEIRERAEKLAEEAKKVAEELEKLAEGDAVLQLYLAEAYALEAAALTIEAVAAAELGASKEELEKIKEKIEEALKKAEEAMKKALAEAKARGRERLVRLIEEARKEFEKLSKAIKELLEQV

SEQ ID NO: 10 - protein, 136 aa, synthetic construct
MREKLEEMLEEFNEVIDELIEITKEDAPELEELRERAEEAVENERLDELEEILDELEVIILEAMFRDLSAAIEMTKAKNDKEKLKELLKQLEELEKRIKELLERAKKRGNKKIIEKLEKLLKEVEKLKKEIEEYLK

SEQ ID NO: 11 - protein, 135 aa, synthetic construct
EEERKEKREKVRAGLKRAIAELPAEVAARCLALLDDASDEEFIEAVLEVLEAMREALVAMAREGRLDAVRRATSHINEVLVDAAELALEKGREYFRRLCLIVCDMMIELIRLEPEQTPELRRIRERLEEIRRRLE

SEQ ID NO: 12 - protein, 90 aa, synthetic construct
GLEEAEKLLEEIFANFEEIVELIKKNIGTERGKKLLKVFVATVDLILARLEQGADLAELAELVKEIAELAKDEEGLEEAEKLVKELTAAR

SEQ ID NO: 13 - protein, 22 aa, synthetic construct
FELLDKLIELLRELIELTREYI

SEQ ID NO: 14 - protein, 18 aa, synthetic construct
LGSVFHHEELAHELGPIP

SEQ ID NO: 15 - protein, 22 aa, synthetic construct
LGSVFHHEESAQRHELHLIEIP

SEQ ID NO: 16 - protein, 13 aa, synthetic construct
SVSEIQLMHNLGK